(19) United States
(12) Patent Application Publication
(10) Pub. No.: US 2006/0024751 A1
(43) Pub. Date: Feb. 2, 2006

(54) Scale-up methods and systems for performing the same

(75) Inventors: Andy May, San Francisco, CA (US); Hany R. Nassef, San Mateo, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Fluidigm Corporation, South San Francisco, CA (US)

(21) Appl. No.: 11/145,892

(22) Filed: Jun. 3, 2005

Related U.S. Application Data
(60) Provisional application No. 60/576,997, filed on Jun. 3, 2004.

(51) Int. Cl. G01N 33/53; G01N 33/48; G06F 19/00

(52) U.S. Cl. 435/7.1; 702/19

(57) ABSTRACT

The present invention provides methods of and systems for translating conditions from a small-volume experiment to a larger-volume experiment.

### CROSS-REFERENCES TO RELATED APPLICATIONS

This application claims the benefit of U.S. Patent Application No. 60/576,997, filed Jun. 3, 2004, the content of which is incorporated herein by reference.

### BACKGROUND OF THE INVENTION

Drug discovery often relies on some knowledge about the structure of a target receptor in designing a drug candidate that might bind to the receptor or influence the binding of another molecule to the target receptor. Protein structures can be determined in some instances by performing X-ray diffraction analysis on crystallized receptors. Traditionally, protein crystallization was achieved by a hit or miss process and brute force screening. Relatively large amounts of target receptor protein were often needed to create a large enough array of crystallization conditions to find at least one condition conducive to crystal formation. Purified protein is often scarce and it may require considerable expense and time to obtain sufficient starting material for crystallization. The use of microfluidic technology to miniaturize crystallization reactions has revolutionized crystallization technology (see Hansen et al in U.S. patent application Ser. No. 11/006,522, filed Dec. 6, 2004; Ser. No. 09/724,784 filed Nov. 28, 2000; Ser. No. 10/997,714 filed Nov. 24, 2004; Ser. No. 60/525,245 filed Nov. 26, 2003 (including Appendix); Ser. No. 09/605,520, filed Jun. 27, 2000; U.S. patent Publication Nos. 20050019794, 20050062196, 20040115731, 20030061687, 20020145231, 20010020636 and International Patent Publication No. WO 01/32930) and allows rapid and efficient determination of optimal crystallization conditions and production of high quality crystals. However, limitations exist because crystals that may result from the screen may not be large enough to permit analysis, e.g., placed into an x-ray beam for diffraction studies. In addition, other reactions or processes carried out in a very small volume may result in a small amount of material. Therefore additional methods for producing larger amounts of material (e.g., larger or more high quality crystals) would be useful.

### BRIEF SUMMARY OF THE INVENTION

In one aspect the present invention provides a method of translating conditions from a small-volume to a larger-volume comprising:

(a) identifying one or more first test level(s) of one or more variable(s) that produce a first performance characteristic in a small volume;

(b) adjusting one or more level with an adjustment factor which factor which is based on the first performance characteristic produced in the small volume and the volume differences between the small volume and the larger volume; thereby producing one or more second test level(s) of one or more variable(s) for producing a second performance characteristic in a larger volume.

In another aspect the present invention provides a method of producing a product in a large-volume comprising:

(a) identifying one or more test level(s) of one or more variable(s) in a small volume that produce a first performance characteristic;

(b) adjusting one or more test level(s) with an adjustment factor which is based on the first performance characteristic produced in the small volume and the volume difference between the small volume and a larger volume; thereby producing one or more second test level(s) of one or more variable(s);

(c) producing a product using said second test level(s) in a larger-volume.

In another aspect the present invention provides method of formulating an adjustment factor for translating conditions from a small-volume to a larger-volume comprising:

(a) identifying one or more test level(s) of one or more variables that produce a first performance characteristic in a small volume for further analysis;

(b) identifying one or more test level(s) of one or more variables that produce a second performance characteristic in a large volume for further analysis;

(c) forming an analytical model for obtaining optimum values of the performance characteristic using the identified test levels of variables; and

(e) formulating an adjustment factor using the analytical model

In another aspect the present invention provides a method of translating crystallization conditions from a small-volume experiment to a larger-volume experiment comprising:

(a) identifying one or more first test level(s) of one or more variable(s) that produce a first crystal property in the small volume;

(b) adjusting one or more test level with an adjustment factor which factor which is based on the first performance characteristic produced in the small volume and the volume differences between the small volume and the larger volume; thereby producing one or more second test level(s) for producing a second crystal property in a larger volume.

In another aspect the present invention provides a method of producing an x-ray quality crystal comprising:

(a) identifying one or more first test level(s) of one or more variable(s) that produce a first crystal property in a small-volume;

(b) adjusting one or more test level(s) with an adjustment factor which is based on the first crystal property produced in the small volume experiment and the volume difference between the small volume experiment and the larger volume experiment;

(c) producing a x-ray quality crystal using said second test level(s) in a larger-volume.

In another aspect the present invention provides a computer implemented method of translating conditions from a small-volume to a larger-volume comprising:

(a) identifying one or more test level(s) of one or more variables that produce a first performance characteristic in a small volume for further analysis;

(b) identifying one or more test level(s) of one or more variables that produce a second performance characteristic in a large volume for further analysis;

(c) recording the values of the test levels, variables and performance characteristics at a particular volume;

(d) forming an analytical model for obtaining optimum values using the recorded values;

(e) and formulating an adjustment factor using the analytical model thereby producing a second set of test levels for producing a second performance characteristic in a larger volume.

In another aspect the present invention provides a database formed by the above method.

In another aspect the present invention provides a system comprising a database, as above, and a computer apparatus having access to said database capable of analyzing the outcome of small-volume and larger volume experiments.

### BRIEF DESCRIPTION OF THE DRAWINGS

FIG. 1 shows examples of crystal density on a X.96 screening chip.

FIG. 2 shows examples of crystal showers in a X.96 screening chip.

FIGS. 3 shows examples of Grade A crystals found in a X.96 screening chip.

FIG. 4 shows examples of Grade B crystals found in a X.96 screening chip.

FIG. 5 shows examples of Grade C crystals found in a X.96 screening chip.

FIG. 6 shows the results of translation of conditions from a X.96 screening chip to a X-ray chip.

FIG. 7 shows the results of translation of conditions from a X.96 screening chip to a X-ray chip.

FIG. 8 shows examples of Grade A crystals with low crystal density from a X.96 screening chip to a X-ray chip.

FIG. 9 shows examples of Grade B crystals with high crystal density from a X.96 screening chip to a X-ray chip.

FIG. 10 is a flow chart illustrating initial condition translation from a screening chip to a X-ray chip based on assessments of crystal density and quality.

FIG. 11 is a flow chart illustrating condition optimization after translation to a X-ray chip.

FIG. 12 is a flow chart illustrating condition translation from a screening chip to macroscopic methods based on assessments of crystal density and quality.

FIG. 13 is a printout of a spreadsheet that represents how the spreadsheet/database may appear and be used.

FIG. 14 shows the results of a crystallization hit obtained using OptiMix-2 reagent 72 (0.4M Potassium Acetate, 0.1M MES pH 5.5, 25% Polyethylene glycol 6000) in the 1.96 chip.

FIG. 15 shows crystals grown in an X-ray chip using the reagents designed by the translation workbook.

FIG. 16 shows the results of a crystallization hit obtained using OptiMix-4 reagent 8 (0.8M Sodium Formate, 0.1M Sodium Citrate pH 5.6, 23% Polyethylene glycol 4000) in the 1.96 chip.

FIG. 17 shows crystals grown in an X-ray chip using the reagents designed by the translation workbook.

FIG. 18 shows the results of a crystallization hit obtained using OptiMix-3 reagent 64 (1M Sodium Malonate, 0.1M Tris pH 8.5) in the 1.96 chip.

FIG. 19 shows crystals grown in a (1 μl+1 μl) sitting drop vapor diffusion experiment using the reagents designed by the translation workbook.

FIG. 20 shows the results of a crystallization hit obtained using OptiMix-3 reagent 18 (2M Ammonium sulphate, 0.1M Hepes pH 7.5) in the 1.96 chip.

FIG. 21 shows crystals grown in a microbatch experiment (1 μl+1 μl) using the reagents designed by the translation workbook.

### DETAILED DESCRIPTION OF THE INVENTION

### DEFINITIONS

As used herein the terms “translate” and “translating” refer to the use of an adjustment factor allows that allows one to produce a desired performance characteristic in a larger-volume experiment from the result obtained in a small-volume experiment.

As used herein, the term “adjustment factor” is a numerical factor which calculates a test value, a set of test values or sampling intervals for a higher volume experiment based on previous results obtained in translating a first performance characteristic into a second performance characteristic.

As used herein the terms “adjust,” “adjusting” and equivalents refer to changing the value of a test level.

As used herein, the term “performance characteristic” describes an output, which may be either qualitative or quantitative, of an experimental method designed to test the performance of a particular system.

As used herein the term “variable” refers to a material of which the quantity can be varied.

As used herein a “level” or “test level” refers to a measurable quantity of a variable and is typically defined in terms of a concentration, a ratio between variables, a rate associated with a variable and the like.

As used herein, the term “condition” describes the set of test levels of variables in a small-volume or large-volume experiment.

As used herein, the term “hit condition” describes the set of test levels of variables in a small-volume or large-volume experiment that product a desired performance characteristic.

As used herein the term “target material” refers to the material being crystallized in a crystallization experiment.

As used herein, the terms “precipitant” or “crystallizing agent” describe a substance that is introduced to a solution of target material to lessen solubility of the target material and thereby induce crystal formation. Crystallizing agents typically include countersolvents in which the target exhibits reduced solubility, but may also describe materials affecting solution pH or materials such as polyethylene glycol that effectively reduce the volume of solvent available to the target material. The term “countersolvent” is used interchangeably with “crystallizing agent”.

As used herein, the term “reagent” refers broadly to any agent used in a reaction, other than the analyte or target (e.g., protein being analyzed/crystallized). Exemplary reagents for crystallizations include, for example, polyethylene glycols, salts, water soluble organic molecules, detergents, and the like.

As used herein, the term “small-volume” means a volume of about 0.1 to about 0.0001 of a larger volume. Preferably the small volume is about 0.1 to about 0.001 of a larger volume, and more preferably about 0.1 to about 0.01 of a larger volume. Preferably the small volume is less than about 70 nL, preferably about 0.5 to about 70 nL and more preferably about 1 to about 10 nL. For most small volume crystallization screening assays, the total volume, including screening solution is approximately 4 to 10 nL. For example, a experiment in a Topaz x.96 chip uses a total of about 0.77nl of protein solution and about 2.7nL of crystallizing reagent. However, the present invention is not limited to any particular volume or range of volumes. Alternative embodiments in accordance with the present invention may utilize total volumes of less than 10 nL, less than 5 nL, less than 2.5 nL, less than 1.0 nL, and less than 0.5 nL.

As used herein, the term “larger-volume” means a volume of about 10 to about 10000 -fold larger than a small-volume. Preferably the larger-volume is about 10 to about 1000 of a small volume, and more preferably about 10 to about 100 of a small-volume. Preferably the larger volume is greater than about 75 nL, preferably about 75 to about 200 nL and more preferably about 75 to about 150 nL.

As used herein, the term “crystal property” describes the performance characteristic of a crystallization experiment. Examples of crystal properties include, but are not limited to crystal quality, crystal density, the quality of an X-ray diffraction pattern collected from a crystal, the size of a crystal.

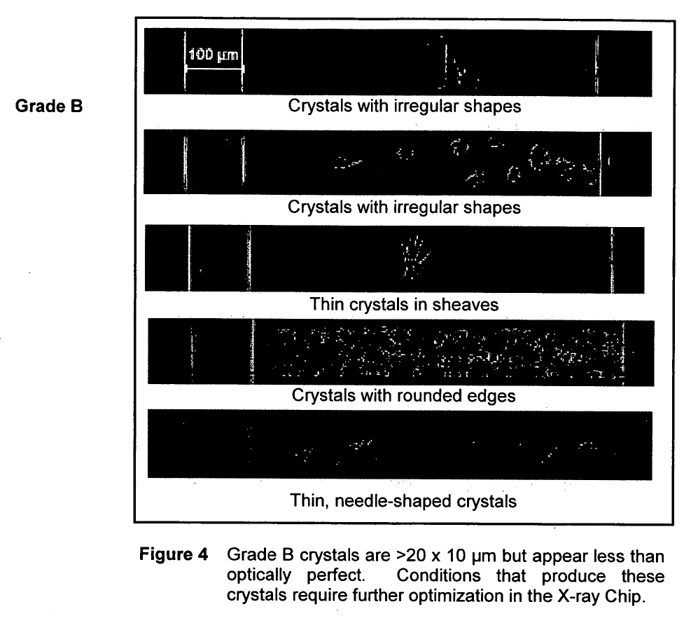

As used herein, the term “crystal quality” is defined in three categories: 1) “Grade A” or “high quality”; 2) “Grade B” or “medium quality” and 3) “Grade C” or “low quality”. Crystal quality is a function of crystal size. Grade A crystals are >20×10 μm and appear optically perfect, with no obvious flaws in the crystal, no satellite crystals, and no signs of potential problems within the crystal lattice. A satellite crystal is a smaller crystal growing on the face of an otherwise perfect crystal. See FIG. 3 for examples of Grade A crystals. Grade B crystals are >20×10 μm but appear less than optically perfect. Examples of imperfections in Grade B crystals include obvious lattice defects on the crystal surface, sheaves of crystals, cracks in the crystal, and satellite crystals. See FIG. 4 for examples of Grade B crystals. Grade C crystals, shown in FIG. 5, are <20×10 μm in size and often appear in crystal showers.

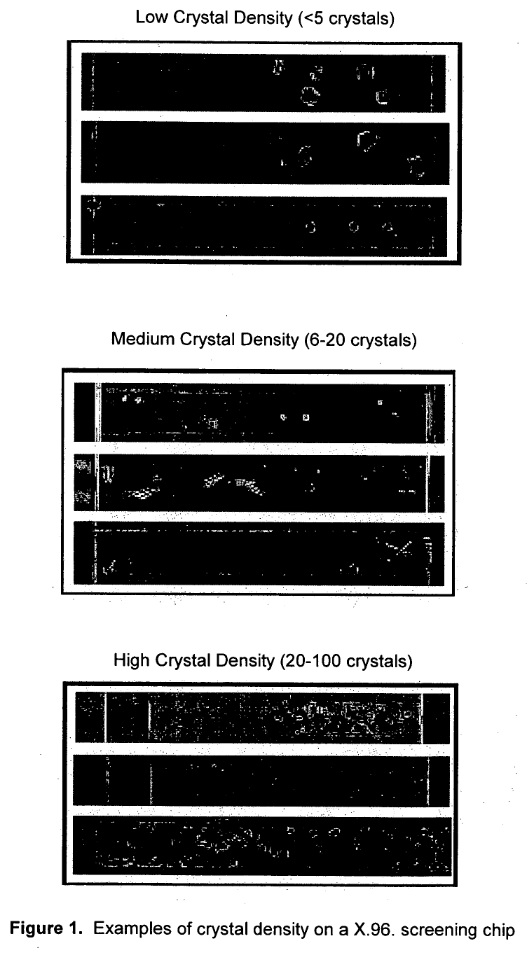

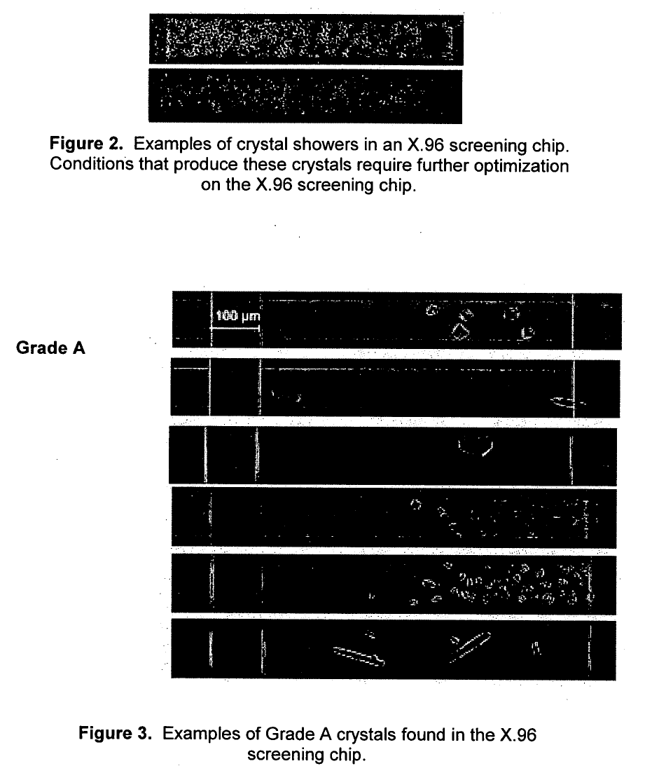

As used herein, the term “crystal density” describes the number of crystals in a device such as 96-well screening chip, as shown in FIG. 1. In one embodiment, the assessment of crystal density is used to set the adjustment factor (ranges and sampling intervals) for translating the low-volume results into the higher-volume experiments. “Low crystal densities” means <6 crystals/protein chamber. “Medium densities” means 6-20 crystals/protein chamber. “High densities means >20-100 crystals. In some cases, the high density refers to a shower of hundreds of small crystals within the device, as shown in FIG. 2.

As used herein, the term “x-ray quality crystal” describes a crystal from which a suitable set of X-ray diffraction data can be obtained.

As used herein, the term “plurality” means at least two. In general a plurality of experiments, compounds, etc., means at least 10, at least about 10², at least about 10³, or at least about 10⁴ different experiments, compounds, etc.

1. Overview

A variety of chemical reactions and processes can be carried out in very small volumes using microfluidic and other nano-technologies. It is often desirable to scale-up a small volume reaction or process so that larger amounts of product can be obtained. In studies in which small volume crystallization reactions have been scaled up it has been discovered that, unexpectedly, simply increasing the volumes used in a crystallization experiment results in changes (e.g., changes in kinetics of crystallization) and the direct replication of the conditions used in the small volume are often not optimized for producing crystals in a scaled-up, larger-volume experiment. Therefore, a need exists for new methods and systems for scaling up conditions resulting from small-volume processes so that they are optimized for larger volumes.

Chemical processes are carried out under a specified condition that affects the outcome of the process. A condition can be described as a set variables each present at a certain level. Variables can be materials in the reaction mixture for which the quantity can be varied to different levels (e.g., expressed as a concentration, ratio of amounts, and the like) or a physical state, such as reaction rate, duration, temperature, changes in temperature, diffusion rate, and the like, which can be varied to different levels (expressed in appropriate units, e.g. degrees centigrade, etc.). For example, a particular protein (e.g., ArnA) can be crystallized using the hanging drop method of vapor diffusion where the condition in which crystallization occurs is at ArnA (6.0 mg/ml, pre-incubated for 30 min on ice with 3 mM UDP-GlcA and 3 mM MgSO4), a protein: precipitant ratio 2.0 ml: 2.0 ml, 0.1M, MES pH 7.0, 10% Ethylene glycol, 9% Polyethylene glycol 8000,14 mM 2-mercaptoethanol and 10 mM ATP, at 16° C. Changing or adjusting a condition can affect the outcome of the reaction or process. For example, changing the concentration of a precipitating agent in a crystallization can affect the number, quality, size, and other characteristics of any resulting crystals. For any particular process particular the condition (i.e., an “optimized condition”) can be identified or selected to give a desired result or performance characteristic. An example of a desired result or performance characteristic in production of protein crystals is production of X-ray quality crystals.

As noted above, the volume also affects the process. A condition that gives a specified result for a process in a small volume may not give the same result in a larger volume. In one aspect, the present invention provides methods for determining a condition for a larger volume that gives the same result as a specified condition in a small volume. The process of determining the condition for a larger volume that gives the same result as a specified condition in a small volume is referred to as “translating” the small volume condition to the large volume condition. This process can also be referred to as “scaling-up.” In translating the condition (e.g., an optimized condition) in a small volume to a larger volume, levels of variables may be changed or adjusted.

In one embodiment, the present invention provides a method of translating conditions from a small-volume to a larger-volume comprising:
(a) identifying one or more test level(s) of one or more variables that produce a first performance characteristic in a small volume;
(b) adjusting one or more test level with an adjustment factor which factor which is based on the first performance characteristic produced in the small volume and the volume differences between the small volume and the larger volume; thereby producing a second set of test levels for producing a second performance characteristic in a larger volume.

In another embodiment, the present invention provides a method of producing a product in a large-volume comprising:
(a) identifying one or more test level(s) of one or more variable(s) in a small volume that produce a first performance characteristic;
(b) adjusting one or more test level(s) with an adjustment factor which is based on the first performance characteristic produced in the small volume and the volume difference between the small volume and a larger volume; thereby producing one or more second test level(s) of one or more variable(s);
(c) producing a product using said second test level(s) in a larger-volume.

It will be apparent upon review of this disclosure that the first performance characteristic and the second performance characteristic are related and may be the same or different characteristics depending on the application. For example, in the case of protein crystallization, the first (small volume) performance characteristic can be small, Grade A crystals and the second (larger volume) performance characteristic can be a larger, Grade A crystal. In any event, both the first performance characteristic and the second performance characteristic are attributes desired by the practitioner (e.g., quality of crystal, correctly refolded protein, amount of protein expressed, and the like.)

As is explained in detail below, the process of (a) identifying one or more test levels of one or more variables that produce a first performance characteristic in a small volume can be conducted by screening a number (preferably a large number) of different conditions and evaluating the performance characteristic associated with each condition. This can be done, for example, using a microfluidic device such as a TOPAZ chip (Fluidigm) to assay output under numerous conditions. If the condition that produces the desired performance characteristic desired (that is, the optimal condition) is described, as discussed above, as a set variables each present at a certain level, the levels of one or more of those variables can be changed in the conditions carried out in the larger volume. A level of a variable used in either a small volume experiment or a larger volume experiment is sometimes referred to as a test level.

A variety of methods can be used to identify an optimal condition for producing a first performance characteristic. Within this embodiment, the present invention provides a method in which a test level of a variable is identified by performing a plurality of small-volume experiments that vary one or more test level(s) of one or more variable(s) to produce a performance characteristic. These experiments may be performed in small-volume and/or large-volume devices. Further these experiments may be performed in series or in parallel by simultaneously introducing the variables at various test levels into a plurality of chambers into the device. Examples of small-volume devices that can be used in the present invention include, but are not limited to a microfluidic array device such as those described generally by Hansen et al in U.S. patent application Ser. No. 11/006,522, filed Dec. 6, 2004; Ser. No. 09/724,784 filed Nov. 28, 2000; Ser. No. 10/997,714 filed Nov. 24, 2004; Ser. No. 60/525,245 filed Nov. 26, 2003 (including Appendix); Ser. No. 09/605,520, filed Jun. 27, 2000; U.S. patent Publication Nos. 20050019794, 20050062196, 20040115731, 20030061687, 20020145231, 20010020636 and International Patent Publication No. WO 01/32930; which are herein incorporated by reference in their entirety for all purposes. Examples of large-volume devices, include but are not limited to a microtiter plate, a vapor diffusion plate, a microbatch plate, a microdialysis chamber, a capillary tube and the like.

As is explained in detail below, the process of (b) adjusting at least one test level based in (a) to produce a second performance characteristic in a larger volume can (and generally does) involve conducting a series of larger-volume assays in which level of at least one variable is adjusted. More often levels of each of several variables is adjusted. In various embodiments, at least 1, 100, 200, 300, 1000, 2000 or more larger-volume assays are conducted. In various embodiments, at least 2, 3, 4, 5, 6 or more variables are adjusted. As discussed below, in an embodiment, the variables are adjusted by a predetermined adjustment factor. An adjustment factor is a numerical factor which calculates a test value, a set of test values or sampling intervals for a higher volume experiment based on previous results obtained in translating a first performance characteristic into a second performance characteristic. The adjustment factor(s) for a protein crystallization experiments are discussed in the next section.

Thus, in another embodiment, the present invention provides a method of formulating an adjustment factor for translating conditions from a small-volume to a larger-volume comprising:
(a) identifying one or more test level(s) of one or more variables that produce a first performance characteristic in a small volume for further analysis;
(b) identifying one or more test level(s) of one or more variables that produce a second performance characteristic in a large volume for further analysis;
(c) forming an analytical model for obtaining optimum values of the performance characteristic using the identified test levels of variables; and
(e) formulating an adjustment factor using the analytical model. In another embodiment steps (a) and/or (b) comprise performing a plurality of experiments that vary one or more test level(s) of one or more variable(s) to produce the performance characteristic.

The following section describes the use of the method in production of crystals in larger volumes. It will also be apparent that the method of the invention has diverse applications in areas such as, in addition to crystallization, combinatorial chemistry, chemical synthesis, chemical formulation, in vitro biological processes and the like. For example, as discussed below, this method can be applied to identification of reaction conditions for enzymatic reactions, protein refolding experiments, chemical formulation for aqueous solubility, cell-free protein expression, and the like, and producing desired products from the processes. The nature or type of variables and performance characteristics produced will depend on the particular application. For illustration, examples of variables for enzyme reactions can include substrate, pH adjusting agents, ionic compounds, enzyme. Examples of performance characteristics for enzymatic reactions may include, but are not limited to, the quantity of product obtained, and the like. For illustration, examples of variables for cell-free protein expression can include: pH, reagent concentrations, incubation temperature, induction time, concentration of induction reagent, energy source, and the like. Examples of performance characteristics for protein expression may include, but are not limited to, the level of active protein expression and the like. For illustration, examples of variables for chemical formulation can include: pH, salt concentration, PEG concentration, organic solvent concentration, mixing time and the like. For illustration, examples of variables for protein refolding can include detergent concentration, salt concentration, salt type, oxidizing reagent, reducing reagent, redox potential, denaturant, chaotrope and the like. Examples of performance characteristics for protein refolding may include, but are not limited to, retention time on a size exclusion column, specific activity of the protein and the like.

2. Crystallization

As mentioned above, small-volume screening experiments are used to identify conditions which produce a crystal, albeit one which is not large enough to collect X-ray diffraction data from. Therefore, it is desirable to produce a larger crystal from that condition. Thus in one embodiment, the invention provides for methods for scaling-up crystallization conditions from a small-volume to a larger-volume. In one embodiment the invention provides a method of translating crystallization conditions from a small-volume to a larger-volume experiment comprising:
(a) identifying one or more first test level(s) of one or more variable(s) that produce a first crystal property in the small volume;
(b) adjusting one or more test level with an adjustment factor which factor which is based on the first performance characteristic produced in the small volume and the volume differences between the small volume and the larger volume; thereby producing one or more second test level(s) for producing a second crystal property in a larger volume.

In another embodiment, the invention provides a method of producing an x-ray quality crystal comprising:
(a) identifying one or more first test level(s) of one or more variable(s) that produce a first crystal property in a small-volume;
(b) adjusting one or more test level(s) with an adjustment factor which is based on the first crystal property produced in the small volume experiment and the volume difference between the small volume experiment and the larger volume experiment;
(c) producing a x-ray quality crystal using said second test level(s) in a larger-volume.
   A. Test levels Variables, and Crystal Properties

As noted previously, the small-volume experiments can be conducted in a variety of devices. Examples of small volume devices that can be used in the experiments of the present invention include, but are not limited to, standard commercially available microfluidic chips such as the TOPAZ™ 1.96 and 4.96 screening chips available from Fluidigm Corp., South San Francisco, Calif., and sparse matrix screening chips Crystal Screen I and Crystal Screen II available from Hampton Research of Aliso Viejo, Calif., and Wizard I and Wizard II available from Emerald Biostructures of Bainbridge Island, Wash. Crystallization experiments conducted with a total volume of less than 200 nl carried in devices utilized in conventional macroscopic methods, such as, but not limited to, a vapor diffusion plate, a microtiter plate, a microbatch plate or a capillary; microfluidic devices such as those described by Zheng et al., JACS. 2003, 125, 11170-11171; Zheng et al, Angewandte Chem. 453974 (2004). The TOPAZ™ 1.96 and 4.96 screening chips, for example, allows the screening of a large array of different test levels of variables for a particular performance characteristic.

Examples of large volume devices that can be used in the crystallization methods of the present invention include, but are not limited to microfluidic devices such as those described by Hansen et al in U.S. patent application Ser. No. 11/006,522, filed Dec. 6, 2004; Ser. No. 09/724,784 filed Nov. 28, 2000; Ser. No. 10/997,714 filed Nov. 24, 2004; Ser. No. 60/525,245 filed Nov. 26, 2003 (including Appendix); Ser. No. 09/605,520, filed Jun. 27, 2000; U.S. patent Publication Nos. 20050019794, 20050062196, 20040115731, 20030061687, 20020145231, 20010020636 and International Patent Publication No. WO 01/32930; screening chips such as the TOPAZ X-ray Preparative Chip; the TOPAZ Growth Chip; microfluidic devices such as those described in Zheng et al., JACS. 2003, 125, 11170-11171; Zheng et al, Angewandte Chem. 453974 (2004); and devices utilized in conventional macroscopic methods, such as, but not limited to, a vapour diffusion plate, a microtiter plate, a microbatch plate or a capillary, or a microdialysis cassette.

1. Target Materials for Crystallization

In one embodiment, crystals resulting from the methods of the present invention can be utilized for x-ray crystallography to determine the three-dimensional molecular structure of the target. Typical targets for crystallization are diverse and the methods and systems of the present invention are particularly suited to crystallizing a variety of targets. A target for crystallization may include, but is not limited to: 1) biological macromolecules (cytosolic proteins, extracellular proteins, membrane proteins, DNA, RNA, and complex combinations thereof), 2) pre- and post-translationally modified biological molecules (including but not limited to, phosphorylated, sulfolated, glycosylated, ubiquitinated, etc. proteins, as well as halogenated, abasic, alkylated, etc. nucleic acids); 3) deliberately derivatized macromolecules, such as heavy-atom labeled DNAs, RNAs, and proteins (and complexes thereof), selenomethionine-labeled proteins and nucleic acids (and complexes thereof), halogenated DNAs, RNAs, and proteins (and complexes thereof), 4) whole viruses or large cellular particles (such as the ribosome, replisome, spliceosome, tubulin filaments, actin filaments, chromosomes, etc.), 5) small-molecule compounds such as drugs, lead compounds, ligands, salts, and organic or metallo-organic compounds, and 6) small-molecule/biological macromolecule complexes (e.g., drug/protein complexes, enzyme/substrate complexes, enzyme/product complexes, enzyme/regulator complexes, enzyme/inhibitor complexes, and combinations thereof). Crystallization targets, particularly those of biological origin, may often be modified to enable crystallization. Such modifications include but are not limited to truncations, limited proteolytic digests, site-directed mutants, inhibited or activated states, chemical modification or derivatization, etc. Such targets are the focus of study for a wide range of scientific disciplines encompassing biology, biochemistry, material sciences, pharmaceutics, chemistry, and physics. However, crystallization in accordance with the present invention is not limited to any particular type of target material.

2. Variables Influencing Crystallization as a Function of their Test Level.

In one aspect of the present invention, test levels of the variables in a small-volume crystallization chambers are systematically varied thereby providing a large number of different crystallization conditions.

3. Chemical Variables Used to Influence Crystallization of the Target

In addition to the change in volume between the chamber size of the small-volume screening experiment and the chamber size of the scaled-up/larger volume preparative experiment, several chemical variables may be used to determine one or more conditions suitable for producing crystals from a given target.

During crystallization screening, a large number of chemical compounds may be employed as variables. These compounds include, but are not limited to, the target, salts, small and large molecular weight organic compounds, buffers, ligands, small-molecule agents, detergents, peptides, crosslinking agents, derivatizing agents, co-crystallizing agents, hydrates, seed materials, and the like. Together, these compounds can be used to vary the ionic strength, pH, solute concentration, the target concentration, and can even be used to modify the target. The desired concentration of these chemicals to achieve crystallization is variable, and can range from nanomolar to molar concentrations. A typical crystallization mix contains a set of fixed, but empirically-determined, types and concentrations of ‘precipitants’, buffers, salts, and other chemical additives (e.g., metal ions, salts, small molecular chemical additives, cryo protectants, etc.). Water is a key solvent in many crystallization trials of biological targets, as many molecules may require hydration to stay active and folded.

‘Precipitating’ agents or ‘precipitants; act to push targets from a soluble to insoluble state, and may work by volume exclusion, changing the dielectric constant of the solvent, charge shielding, and molecular crowding. Precipitating agents include, but are not limited to, non-volatile salts, high molecular weight polymers, polar solvents, aqueous solutions, high molecular weight alcohols, divalent metals.

Precipitating compounds, include large and small molecular weight organics, as well as certain salts. Water itself can act in a precipitating manner for samples that require a certain level of ionic strength to stay soluble. Many precipitants may also be mixed with one another to increase the chemical diversity of the crystallization screen.

A nonexclusive list of salts which may be used as precipitants is as follows: Tartrate (Li, Na, K, Na/K, NH₄); Phosphate (Li, Na, K, Na/K, NH₄); Acetate (Li, Na, K, Na/K, Mg, Ca, Zn, NH₄); Formate (Li, Na, K, Na/K, Mg, NH₄); Citrate (Li, Na, K, Na/K, NH₄); Chloride (Li, Na, K, Na/K, Mg, Ca, Zn, Mn, Cs, Rb, NH₄); Sulfate (Li, Na, K, Na/K, NH₄); Malate (Li, Na, K, Na/K, NH₄) and Glutamate (Li, Na, K, Na/K, NH₄.).

A nonexclusive list of organic materials which may be used as precipitants is as follows: PEG 400; PEG 1000; PEG 1500; PEG 2k; PEG 3350; PEG 4k; PEG 6k; PEG 8k; PEG 10k; PEG 20k; PEG-MME 550; PEG-MME 750; PEG-MME 2k; PEG-MME 5k; PEG-DME 2k; Dioxane; Methanol; Ethanol; 2-Butanol; n-Butanol; t-Butanol; Jeffamine M-600; Isopropanol; 2-methyl-2,4-pentanediol; 1,6 hexanediol.

Solution pH can be varied by the inclusion of buffering agents. Typical pH ranges for biological materials lie anywhere between values of 3.5-10.5 and the concentration of buffer, generally lies between 0.01 and 0.25 M. The methods and systems described in this document are readily compatible with a broad range of pH values, particularly those suited to biological targets.

A nonexclusive list of possible buffers is as follows: Na-Acetate; HEPES; Na-Cacodylate; Na-Citrate; Na-Succinate; Na-K-Phosphate; PIPES; TRIS; TRIS-Maleate; Imidazole-Maleate; BisTrisPropane; CAPSO; CHAPS; CHES; MES and imidazole.

Additives are small molecules that affect the solubility and/or activity behavior of the target. Such compounds can speed crystallization screening or produce alternate crystal forms of the target. Additives can take nearly any conceivable form of chemical, but are typically mono and polyvalent salts (inorganic or organic), enzyme ligands (substrates, products, allosteric effectors), chemical crosslinking agents, detergents and/or lipids, heavy metals, organo-metallic compounds, trace amounts of precipitating agents, and small molecular weight organics.

The following is a nonexclusive list of possible additives: 2-Butanol; DMSO; Hexanediol; Ethanol; Methanol; Isopropanol; sodium fluoride; potassium fluoride; ammonium fluoride; lithium chloride anhydrous; magnesium chloride hexahydrate; sodium chloride; Calcium chloride dihydrate; potassium chloride; ammonium chloride; sodium iodide; potassium iodide; ammonium iodide; sodium thiocyanate; potassium thiocyanate; lithium nitrate; magnesium nitrate hexahydrate; sodium nitrate; potassium nitrate; ammonium nitrate; magnesium formate; sodium formate; potassium formate; ammonium formate; lithium acetate dihydrate; magnesium acetate tetrahydrate; zinc acetate dihydrate; sodium acetate trihydrate; calcium acetate hydrate; potassium acetate; ammonium acetate; lithium sulfate monohydrate; magnesium sulfate heptahydrate; sodium sulfate decahydrate; potassium sulfate; ammonium sulfate; di-sodium tartate dihydrate; potassium sodium tartrate tetrahydrate; di-ammonium tartrate; sodium dihydrogen phosphate monohydrate ; di-sodium hydrogen phosphate dihydrate; potassium dihydrogen phosphate; di-potassium hydrogen phosphate; ammonium dihydrogen phosphate; di-ammonium hydrogen phosphate; tri-lithium citrate tetrahydrate; tri-sodium citrate dihydrate; tri-potassium citrate monohydrate; di-ammonium hydrogen citrate; barium chloride; cadmium chloride dihydrate; cobaltous chloride dihydrate; cupric chloride dihydrate; strontium chloride hexahydrate; yttrium chloride hexahydrate; ethylene glycol; Glycerol anhydrous; 1,6 hexanediol; MPD; polyethylene glycol 400; trimethylamine HCl; guanidine HCl; urea; 1,2,3-heptanetriol; benzamidine HCl; dioxane; ethanol; iso-propanol; methanol; sodium iodide; L-cysteine; EDTA sodium salt; NAD; ATP disodium salt; D(+)-glucose monohydrate; D(+)-sucrose; xylitol; spermidine; spermine tetra-HCl; 6-aminocaproic acid; 1,5-diaminopentane di-HCl; 1,6-diaminohexane; 1,8-diaminooctane; glycine; glycyl-glycyl-glycine; hexaminecobalt trichloride; taurine; betaine monohydrate; polyvinylpyrrolidone K15; non-detergent sulfo-betaine 195; non-detergent sulfo-betaine 201; phenol; DMSO; dextran sulfate sodium salt; jeffamine M-600; 2,5 Hexanediol; (±)-1,3 butanediol; polypropylene glycol P400; 1,4 butanediol; tert-butanol; 1,3 propanediol; acetonitrile; gamma butyrolactone; propanol; ethyl acetate; acetone; dichloromethane; n-butanol; 2,2,2 trifluoroethanol; DTT; TCEP; nonaethylene glycol monododecyl ether, nonaethylene glycol monolauryl ether,; polyoxyethylene (9) ether; octaethylene glycol monododecyl ether, octaethylene glycol monolauryl ether,; polyoxyethylene (8) lauryl ether; Dodecyl-β-D-maltopyranoside; Lauric acid sucrose ester; Cyclohexyl-pentyl-β-D-maltoside; Nonaethylene glycol octylphenol ether; Cetyltrimethylammonium bromide; N, N-bis(3-D-gluconamidopropyl)-deoxycholamine; Decyl-β-D-maltopyranoside; Lauryldimethylamine oxide; Cyclohexyl-pentyl-β-D-maltoside; n-Dodecylsulfobetaine, 3-(Dodecyldimethylammonio)propane-1-sulfonate; Nonyl-β-D-glucopyranoside; Octyl-β-D-thioglucopyranoside, OSG; N,N-Dimethyldecylamine-β-oxide; Methyl-6-O-(N-heptylcarbamoyl)-a-D-glucopyranoside; Sucrose monocaproylate; n-Octanoyl-β-D-fructofuranosyl-a-D-glucopyranoside; Heptyl-13-D-thioglucopyranoside; Octyl-β-D-glucopyranoside, OG; Cyclohexyl-propyl-β-D-maltoside; Cyclohexylbutanoyl-N-hydroxyethylglucamide; n-decylsulfobetaine, 3-(Decyldimethylammonio)propane-1-sulfonate; Octanoyl-N-methylglucamide, OMEGA; Hexyl-β-D-glucopyranoside; Brij 35; Brij 58; Triton X-114; Triton X-305; Triton X-405; Tween 20; Tween 80; polyoxyethylene(6)decyl ether; polyoxyethylene(9)decyl ether; polyoxyethylene(10)dodecyl ether; polyoxyethylene(8)tridecyl ether; Isopropyl-β-D-thiogalactoside; Decanoyl-N-hydroxyethylglucamide; Pentaethylene glycol monooctyl ether; 3-[(3-cholamidopropyl)-dimethylammonio]-1-propane sulfonate; 3-[(3-Cholamidopropyl)-dimethylammonio]-2-hydroxy-1-propane sulfonate; Cyclohexylpentanoyl-N-hydroxyethylglucamide; Nonanoyl-N-hydroxyethyglucamide; Cyclohexylpropanol-N-hydroxyethylglucamide; Octanoyl-N-hydroxyethylglucamide; Cyclohexylethanoyl-N-hydroxyethylglucamide; Benzyldimethyldodecyl ammonium bromide; n-Hexadecyl-β-D-maltopyranoside; n-Tetradecyl-β-D-maltopyranoside; n-Tridecyl-β-D-maltopyranoside; Dodecylpoly(ethyleneglycoether)n; n-Tetradecyl-N,N-dimethyl-3-ammonio-1-propanesulfonate; n-Undecyl-β-D-maltopyranoside; n-Decyl-β-D-thiomaltopyranoside; n-dodecylphosphocholine; a-D-glucopyranoside, β-D-fructofuranosyl monodecanoate, sucrose mono-caprate; 1-s-Nonyl-β-D-thioglucopyranoside; n-Nonyl-β-D-thiomaltoyranoside; N-Dodecyl-N,N-(dimethlammonio)butyrate; n-Nonyl-β-D-maltopyranoside; Cyclohexyl-butyl-β-D-maltoside; n-Octyl-β-D-thiomaltopyranoside; n-Decylphosphocholine; n-Nonylphosphocholine; Nonanoyl-N-methylglucamide; 1-s-Heptyl-β-D-thioglucopyranoside; n-Octylphosphocholine; Cyclohexyl-ethyl-β-D-maltoside; n-Octyl-N,N-dimethyl-β-ammonio-1-propanesulfonate; Cyclohexyl-methyl-β-D-maltoside.

Another variable influencing crystal growth is the presence or absence of a seed crystal. Introduction of a seed crystal to the target solution can greatly enhance crystal formation by providing a template to which molecules in solution can align. Where no seed crystal is available, embodiments of crystallization methods and systems in accordance with the present invention may utilize other structures to perform a similar function.

Co-crystallization generally describes the crystallization of a target with a secondary factor that is a natural or non-natural binding partner. Such secondary factors can be small, on the order of about 10-1000 Da, or may be large macromolecules. Co-crystallization molecules can include but are not limited to small-molecule enzyme ligands (substrates, products, allosteric effectors, etc.), small-molecule drug leads, single-stranded or double-stranded DNAs or RNAs, complement proteins (such as a partner or target protein or subunit), monoclonal antibodies, and fusion-proteins (e.g., maltose binding proteins, glutathione S-transferase, protein-G, or other tags that can aid expression, solubility, and target behavior).

Cryosolvents are agents that stabilize a target crystal to flash-cooling in a cryogen such as liquid nitrogen, liquid propane, liquid ethane, or gaseous nitrogen or helium (all at approximately 100-120° K.) such that crystal becomes embedded in a vitreous glass rather than ice. Any number of salts or small molecular weight organic compounds can be used as a cryoprotectant, and typical ones include but are not limited to: MPD, PEG-400 (as well as both PEG derivatives and higher molecular-weight PEG compounds), glycerol, sugars (xylitol, sorbitol, erythritol, sucrose, glucose, etc.), ethylene glycol, alcohols (both short- and long chain, both volatile and nonvolatile), LiOAc, LiCl, LiCHO₂, LiNO₃, Li₂SO₄, Mg(OAc)₂, NaCl, NaCHO₂, NaNO₃, etc.

Many of these chemicals can be obtained in predefined screening kits from a variety of vendors, including but not limited to Fluidigm Corp. of South San Francisco, Calif.; Hampton Research of Laguna Niguel, Calif.; Emerald Biostructures of Bainbridge Island, Wash. and Jena BioScience of Jena, Germany, that allow the researcher to perform both ‘sparse matrix’ and ‘grid’ screening experiments or assays. Sparse matrix screens attempt to randomly sample as much of precipitant, buffer, and additive chemical space as possible with as few conditions as possible. Grid screens typically consist of systematic variations of two or three parameters against one another (e.g., precipitant concentration vs. pH). Screens can also be formulated by individual researchers, and may include, but are not limited to, experimental design methods such as randomization, incomplete factorial, complete factorial, response surface methods, mixture designs, space-filling designs. All of these types of screens have been employed with success in crystallization trials, and the majority of chemicals and chemical combinations used in these screens are compatible with the methods and systems in accordance with embodiments of the present invention.

4. Test Levels

In the methods considered here, the condition of supersaturation which influences nucleation is achieved through the manipulation of the test levels of the chemical variables. Such test levels include but are not limited to: 1) the relative and absolute concentrations of the chemical variables, 2) the equilibration dynamics of the chemical variables, 3) the nucleation rates of the target, and the like.

a. Variations in Concentration

Gross volumes of crystallization experiments or assays can be of any conceivable value, from the picoliter to milliliter range. Typical values ranges may include but are not limited to about 0.1 to about 10,000 nL. Typical values may include but are not limited to: 0.1, 0.2, 0.25, 0.4, 0.5, 0.75, 1, 2, 4, 5, 10, 15, 20, 25, 30, 35, 40, 45, 50, 60, 70, 75, 80, 90, 100, 125, 150, 175, 200, 225, 250, 275, 300, 350, 400, 450, 500, 550, 600, 700, 750, 800, 900, 1000, 1100, 1200, 1250, 1300, 1400, 1500, 1600, 1700, 1800, 1900, 2000, 2250, 2500, 3000, 4000, 5000, 6000, 7000, 7500, 8000, 9000, and 10000 nL.

Crystallization chemical concentration can lie in a range of values and is an important variable in crystallization screening. Typical ranges of concentrations can be anywhere from <0.5 mg/ml to >100 mg/ml, with most commonly used values between 5-30 mg/ml. For example, precipitating compounds are used from under 1% to upwards of 40% concentration, or from <0.5M to greater than 4M concentration.

Target concentration, like crystallization chemical concentration, can lie in a range of values and is an important variable in crystallization screening. Typical ranges of concentrations can be anywhere from <0.5 mg/ml to >100 mg/ml, with most commonly used values between 5-30 mg/ml, depending on the volume of the experimental chamber. For example, if the volume of a chamber on a larger-volume device is 75 nL, the concentration of the protein sample used is preferably at least 8 mg/mL. Use of lower concentrations of protein may limit the size of the protein crystal that is grown in the device. As a crystal forms, it acts as a sink to target material available in solution, to the point where the amount of target material remaining in solution may be inadequate to sustain continued crystal growth. Therefore, in order to grow sufficiently large crystals it may be necessary to provide additional target material during the crystallization process.

The ratios of a target to the variables in the crystallization solution can also constitute an important variable in crystallization screening and optimization. These ratios can be of any of a variety of values, but may range from 1:100 to 100:1 target:crystallization solution and are typically in the range of 1:4 to 4:1 target: crystallization-solution. Typical target: crystallization-solution or crystallization-solution: target ratios may include but are not limited to: 1:100, 1:90, 1:80, 1:70, 1:60, 1:50, 1:40, 1:30, 1:25, 1:20, 1:15, 1:10, 1:9, 1:8, 1:7.5, 1:7, 1:6, 1:5, 1:4, 1:3, 1:2.5, 1:2, 1:1, 2:3, 3:4, 3:5, 4:5, 5:6, 5:7, 5:9, 6:7, 7:8, 8:9, 9:10.

b. Variations in Rates

Still another variable is the ability to control solution equilibration rates. Crystal growth is often very slow, and no crystals will be formed if the solution rapidly passes through an optimal concentration on the way to equilibrium. It may therefore be advantageous to control the rate of equilibration and thereby promote crystal growth at intermediate concentrations of variables. Non limiting examples include reducing the concentration of one or more of the variables/crystal inducing agents in the crystallization reaction solution, altering the pH of the reaction, altering the manner in which the reagent and protein are introduced in the situation of free interface diffusion, and the like. In conventional approaches to crystallization, slow-paced equilibrium is achieved using such techniques as vapor diffusion, slow dialysis, and very small physical interfaces.

Crystallization devices such as those disclosed by Hansen et al in U.S. patent application Ser. No. 11/006,522, filed Dec. 6, 2004; Ser. No. 09/724,784 filed Nov. 28, 2000; Ser. No. 10/997,714 filed Nov. 24, 2004; Ser. No. 60/525,245 filed Nov. 26, 2003 (including Appendix); Ser. No. 09/605,520, filed Jun. 27, 2000; U.S. patent Publication Nos. 20050019794, 20050062196, 20040115731, 20030061687, 20020145231, 20010020636 and International Patent Publication No. WO 01/32930 allow for control over the rate of solution equilibrium. In systems adjusting the concentration of variables (“metering”) by volume exclusion, an overlying membrane can be repeatedly deformed, with each deformation giving rise to the introduction of additional amount of a variable. In systems that meter crystallizing agent by volume entrapment, the valves separating sample from a variable may be opened for a short time to allow for partial diffusive mixing, and then closed to allow chamber equilibration at an intermediate concentration. The process is repeated until the final concentration is reached. Either the volume exclusion or entrapment approaches enables a whole range of intermediate concentrations to be screened in one experiment utilizing a single reaction chamber.

In a particularly preferred embodiment, free interface diffusion between a chamber containing a crystallization variable and a chamber containing a solution having a target therein, wherein the two chambers are in fluid communication through one or more fluid channels, and wherein one or more of the channels may be further in fluid control by a valve associated therewith, wherein the valve is opened and closed at selected intervals to regulate the rate at which diffusion or equilibration between the chambers of one or more of the components.

The manipulation of solution equilibrium over time also exploits differential rates of diffusion of macromolecules such as proteins versus much smaller variables such as salts. As large protein molecules diffuse much more slowly than the salts, rapidly opening and closing interface valves allows the concentration of crystallizing agent to be significantly changed, while at the same time very little sample is lost by diffusion into the larger volume of crystallizing agent. Moreover, as described above, many devices described readily allow for introduction of different variables at different times to the same reaction chamber. This allows for crystallization protocols prescribing changed crystallization conditions over time.

Control over changed crystallization conditions may also result from a variety of techniques, including but not limited to cross-channel injection into a matrix of junctions defined by intersecting orthogonal flow channels. While the above description has focused upon diffusion of a single variable, gradients of two or more of variables which do not interact with each other may be created simultaneously and superimposed to create an array of concentration conditions.

5. Other Factors Influencing Crystallization

While the above methods describe altering the environment of the target material through introduction of volumes of an appropriate variable, many other factors are relevant to crystallization. Such additional factors include, but are not limited to, temperature, pressure, gravity, the length of time allowed for crystallization, the surface in which the crystallization occurs. Other factors may be involved with the scaling up of crystallization reaction from small-volumes to larger volumes.

Preferably the same temperature, pressure, gravity, time and surface conditions are used in the setup and incubation in the larger-volume device as those used in the small-volume device. When conducting experiments or assays based on two different crystallization conditions within a single device, conditions should preferably be chosen that produce crystals within a similar time frame. This assures that crystals that grow in the larger-volume device are ready to be extracted at the same time.

6. Crystal Properties

A crystal property is a performance characteristic of a crystallization experiment. How one translates a “hit condition” from a small-volume experiment to a larger-volume experiment, depends on the particular crystal property desired, therefore, the desired crystal property must be assessed. Crystal properties include, but are not limited to, crystal number/density, crystal quality, crystal size and quality of X-ray diffraction data. When the second crystal property is an x-ray quality crystal, the first crystal properties preferably include the number or density and size or quality of the crystals produced by a particular small-volume “hit” condition.

a. Crystal Number/Density

Crystal density describes the number of crystals in a small-volume device such as 96-well screening chip, as shown in FIG. 1. In one embodiment, the assessment of crystal density is used to set the adjustment factor (ranges and sampling intervals) for translating the small-volume results into the larger-volume experiments or assays. “Low crystal densities” means <5 crystals/protein chamber. “Medium densities” means 6-20 crystals/protein chamber. “High densities means >20-100 crystals. In some cases, the high density refers to a shower of hundreds of small crystals within the device, as shown in FIG. 2.

b. Crystal Quality and Size

For purposes of translating crystallization conditions, we define three categories of crystal quality and size: Grades A, B, and C. Grade A crystals are >20×10 μm and appear optically perfect, with no obvious flaws in the crystal, no satellite crystals, and no signs of potential problems within the crystal lattice. A satellite crystal is a smaller crystal growing on the face of an otherwise perfect crystal. See FIG. 3 for examples of Grade A crystals.

Grade B crystals are >20×10 μm but appear less than optically perfect. Examples of imperfections in Grade B crystals include obvious lattice defects on the crystal surface, sheaves of crystals, cracks in the crystal, and satellite crystals. Another way of thinking about the difference between the A and B categories is that a Grade B crystal may further optimization, while a Grade A crystal does not. See FIG. 4 for examples of Grade B crystals.

Grade C crystals, shown in FIG. 5, are <20×10 μm in size and often appear in crystal showers. Conditions that produce these crystals require further optimization in a small-volume experiment before attempting translation to a larger-volume experiment.

c. Combining Crystal Density and Quality Assessments

These assessments of crystal density and crystal quality guide the test levels of variables for translation of the small-volume experiments or assays to the larger-volume experiment. For a small-volume hit that generates low crystal density, it is assumed that when volume is scaled up the crystal density will remain low. At medium density, however, the number of nucleation events must be reduced as experiment volume increases. As a result, the variable concentrations or rates may need to be significantly reduced from those in the small-volume experiment.

For grade A crystals, it is assumed that: the composition of the crystallization reagent in the small-volume experiment is close to optimal for crystal growth; and larger-volume experiments or assays are designed to replicate that composition. For grade B crystals, the components of the crystallization reagent are not fully optimized for crystal growth. To improve the crystal quality during translation of conditions that produce grade B crystals, experiments or assays sample increased variations around those conditions.

Assessments of crystal properties and the success or failure of a particular crystallization experiment may be done by direct visual inspection via an optical microscope via birefringence of polarized light or can be done using a detector in conjunction with image analysis software, or by x-ray diffraction analysis. Therefore devices may use transparent materials such as polystyrene or silanized glass to allow for visualization. Devices may also use materials with low X-ray absorption coefficients for assessment of crystal quality by the quality of X-ray diffraction data. Birefringence may be difficult to judge in some materials, such as plastics, as they are birefringent, interfering with sample assessment. Automated scanning systems can be used which digitize and port images into scanning software for visualization. It should also be possible to use methods such as light scattering to detect the protein aggregates on smaller length scales. Examples of automated detection and image analysis software include, but are not limited to, the TOPAZ™ AutoInspeX Workstation and Autoprocessor software.

B. Translating Conditions From The Small-Volume Experiment

Once a particular condition is identified which yields a crystal that may be useful in x-ray diffraction, it is desirable to produce a larger crystal from that condition. In one embodiment, it is assumed that the initial crystallization conditions obtained in the small-volume experiment contain the optimal variables to induce crystallization of the target of interest in a larger-volume device. However, simply repeating the same reaction in a larger volume does not necessarily yield the same result. Because, increasing the volumes used in a crystallization experiments or assays results in changes to the kinetics of crystallization; the direct replication of the crystallization condition in the small-volume experiment is not optimal for producing crystals in a larger-volume, scaled-up experiment. Indeed, it is most likely that the larger volume reaction performed with conditions identical to the screening experiment would yield more crystals of the same size of those produced during the screening experiment. The rate of crystal nucleation is the critical parameter that needs to be controlled in translation from a small-volume experiment to a larger-volume experiment. Results from initial translation assays suggest that the rate of nucleation within a crystallization experiment is volume-dependent. For example, if a successful screening hit condition produces 100 crystals in the 0.75-nL small-volume device, then within the larger 75 nL volume device (a 100-fold increase in volume), the same conditions might be expected to yield 10,000 crystals.

To overcome this problem, the invention provides, in one embodiment, for adjusting the reaction conditions with an adjustment factor to limit the number of crystal initiation sites within the reaction chamber of the larger-volume experiment. For example, if the small-volume screening experiment yielded a low rate of nucleation, the larger-volume, scaled up reaction would not be as diluted downward as compared to if the screening reaction yielded a high rate of nucleation. Preferably the number of crystal nucleations in the larger-volume experiment is below one hundred initiations, and more preferably below ten initiations, and most preferably just one initiation. By limiting the number of crystal initiations in the larger-volume experiment, the target/protein remaining in solution in the larger volume may then be used to “feed” the growing few crystals before becoming relatively depleted from the surrounding fluid medium thus resulting in a larger crystal being formed.

Closely related to the problem of volume translation is determining the adjustment factor. Since it may be difficult to determine, a priori, which conditions will induce crystallization, a screening method which systematically samples as many conditions as possible is used to explore “condition/volume space. This can be accomplished by conducting a plurality of assays, through which “condition/volume space” is sampled during the evolution of each assay over time. The methods described herein allow for the combinatorial mixing of multiple variables at multiple test levels with a particular target sample. The ability of such devices to access a vast number of chemical conditions, and to accurately dispense and mix fluids on the picoliter scale makes detailed characterization of scale-up behavior both possible and practical. Automation of metering, mixing and data acquisition allows for thousands of crystallization experiments to be executed with little or no need for user intervention. In each experiment a unique mixture of the variables and the target sample is produced. In this way a condition set may be generated which allows for the generation of an adjustment factor. From these conditions sets in small and larger-volumes an analytical model can be formed for obtaining optimum values of the test levels of the variables which provide the desired performance characteristic. Using the analytical model the adjustment factor can be formulated.

In one embodiment, formulation of the adjustment factor and translation of small-volume conditions to larger volume conditions is done using a computer. Thus in one aspect the invention provides a computer implemented method of translating conditions from a small-volume to a larger-volume comprising:
(a) identifying one or more test level(s) of one or more variables that produce a first performance characteristic in a small volume for further analysis;
(b) identifying one or more test level(s) of one or more variables that produce a second performance characteristic in a large volume for further analysis;
(c) recording the values of the test levels, variables and performance characteristics at a particular volume;
(d) forming an analytical model for obtaining optimum values using the recorded values; and
(e) formulating an adjustment factor using the analytical model thereby producing a second set of test levels for producing a second performance characteristic in a larger volume. In another embodiment, the invention provides a database formed by the above method. In another embodiment, the invention provides a system comprising the above database and a computer apparatus having access to said database capable of analyzing the outcome of small-volume and larger volume experiments.

Thus detailed knowledge of protein crystallization behavior in small and large volumes provides an empirical basis for the formulation of an adjustment factor which increases the likelihood of translating crystallization from small-volume conditions into larger-volume experiments or assays which provide a desired performance characteristic such as an x-ray quality crystal. From this thorough characterization of the scale-up behavior, adjustment factors have been developed for crystallization screens of targets. Accordingly, one aspect the invention provides an empirically derived adjustment factor, the use of which is illustrated as follows.

1. Flow Charts for Translation

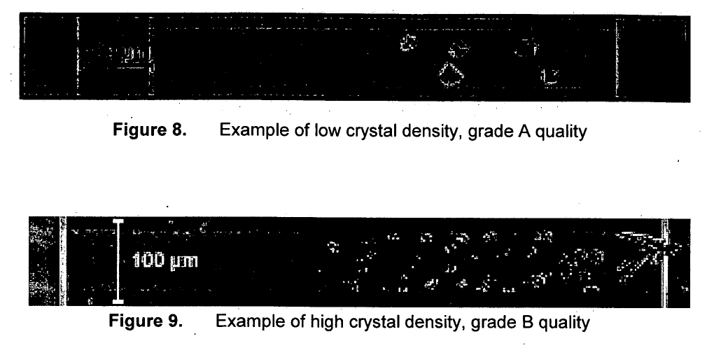

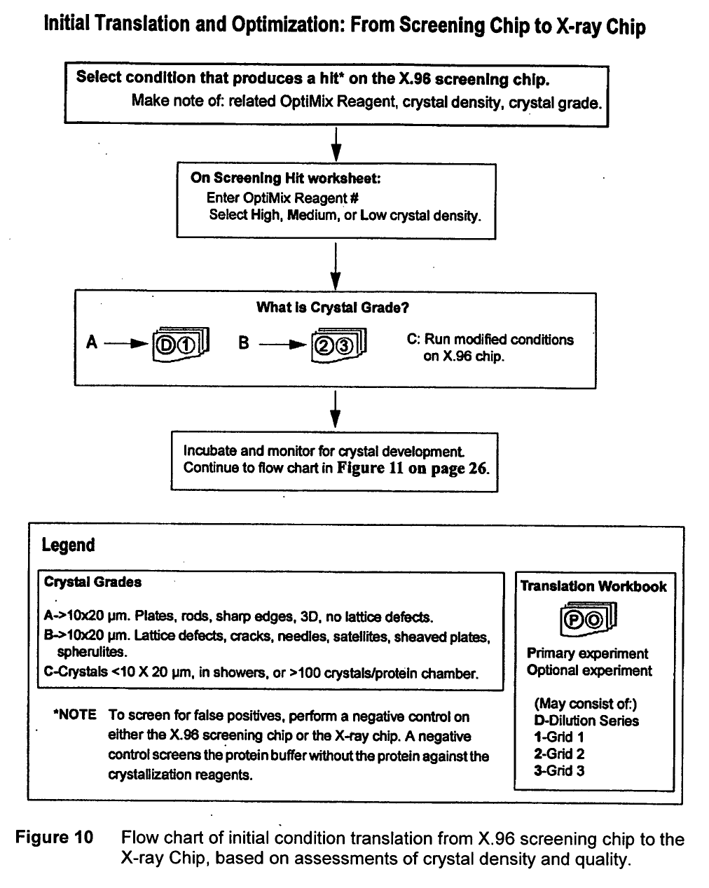

The Flow Charts of FIGS. 10-12 illustrate how one can use the adjustment factor based on the different crystal properties described above to develop experiment scenarios that support translation from a small-volume experiment to a larger volume experiment. For example, if a small volume experimental condition results in a grade A crystal quality and low crystal density, as shown in FIG. 8, a large-volume experiment which varies salt concentration in fine intervals of 2.5% of starting concentration, and/or a dilution series which varies precipitant concentration in 2.5% intervals would give the highest probability of obtaining X-ray quality crystals. As shown in FIG. 9, a hit condition with a larger number of small, lower quality crystals (the crystal density would be high, with grade B crystal quality), as shown in FIG. 9, a set of larger-volume assays that provides larger variation of pH values (e.g. between about 4.0 to 9.5), along with larger variations (5% intervals of starting reagent concentrations) in the concentrations of the salt and precipitant would give the highest probability of obtaining X-ray quality crystals.

FIG. 10 and FIG. 11 show flow charts of experimental paths for different combinations of crystal density and crystal quality. The flow chart in FIG. 10 provides a decision path for the initial part of a translation experiment: what to run in a large-volume device, such as an X-ray Chip, after an initial hit is obtained in a small-volume device, such as a X.96 screening chip.

Each flowchart, also suggests additional experiments or assays if a greater number of experiments can be run, for example on both halves of a 96-well X-ray Chip. The primary experiment, shown in bold, is used if only one experiment is to be run and the optional experiment is used if additional experiments or assays can be run to increase the chances of successful translation. For example, if the crystal is grade A, the flowcharts suggest that the primary experiment be a dilution series, with an optional experiment of Grid 1. For grade B crystals, the suggest primary experiment is Grid 2, with an optional Grid 3. These assays are explained in greater detail below.

When the reagent only contains two components, the recommended primary experiment may change. For example, if the reagent contains either a salt or a precipitant combined with a buffer, Grid 1 becomes redundant with the dilution series; Grid 2 becomes redundant with Grid 3. For such cases the dilution series and Grid 3 may be run together. If the crystals are grade A, then the dilution series is the primary experiment. If they are grade B, then Grid 3 becomes the primary experiment. If the crystallization reagent does not contain a buffer, then Grid 2 becomes redundant with Grid 1; Grid 1 becomes the primary grid for grade B crystals.

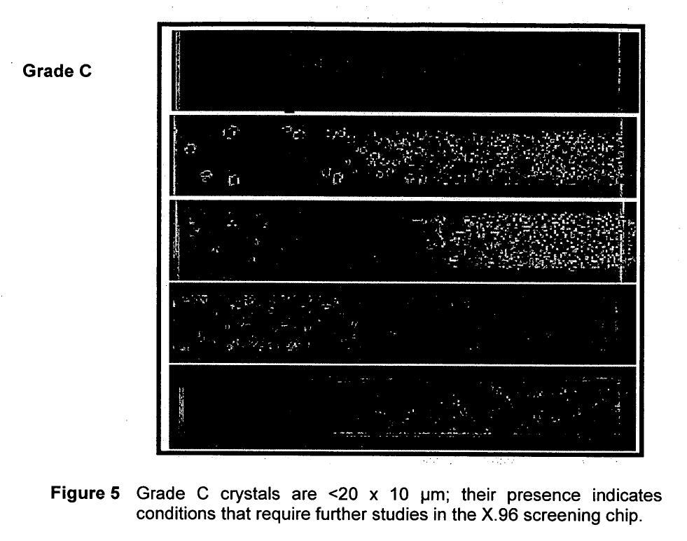

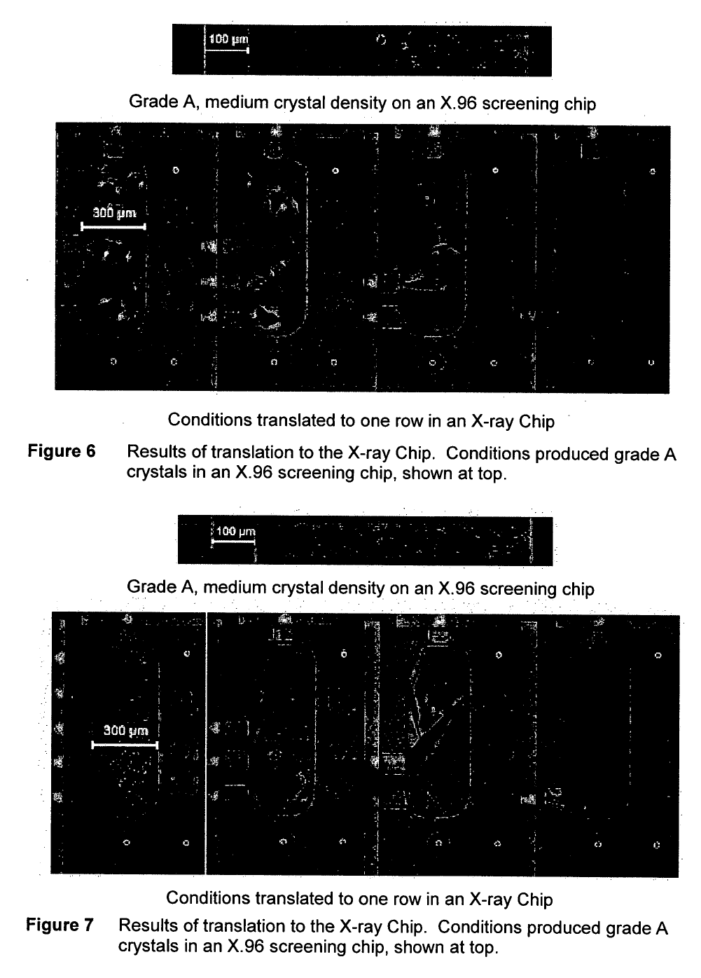

FIG. 6 and FIG. 7 show examples of successful translation of conditions that were initially found on a X.96 screening chip and then run on a X-ray Chip. Comparison of this method to straight-scale up showed an improvement from 30% to 70% in the success rate of translation The methods of the present invention therefore help the practitioner avoid, 1) useless experiments on chemicals that do not alter crystallization significantly (and hence will not produce crystals), and 2) useless experiments that are either too supersaturated and result only in protein aggregate, or are too undersaturated and result in the protein remaining in solution.

2. Using The System

In one embodiment, the translation between conditions identified in a small volume screening outlined above is done by a spreadsheet program having embedded therein the adjustment factor which calculates adjustments for differences in volumes between the chamber used in the small-volume, screening-type experiment and the chamber used in the larger volume, scale-up experiment. The spreadsheet provides a series of experiments to be performed wherein for example, the scale up experiments explore a dilution series of the crystallization reagent, variations in pH, and/or variations in diffusion rates.

In another embodiment, the present invention provides a method for using a system of the present invention comprising:
(a) identifying in the database one or more test level(s) of one or more variable(s) in a small volume using the computer;
(b) obtaining one or more second test level(s) of one or more variable(s) for a larger-volume;
(c) producing a product using said second test level(s) in a larger-volume. An example of a database is described below. This database is may designed in a spreadsheet program for example, Microsoft® Excel. The spreadsheet uses as inputs a value that uses an estimate of crystal density and the variables from reagents such as OptiMix™ Reagents (Fluidigm Corp.) from a “hit” condition as inputs to calculate grids for simultaneously translating and optimizing crystallization conditions from small-volume device, such as a TOPAZ™ X.96 screening chip (Fluidigm Corp.) to a large-volume device, such as the TOPAZ X-ray Chip (Fluidigm Corp.) or to conventional methods.

In one embodiment, the database may comprise several worksheets as shown in Table 1.

TABLE 1
Worksheets in Database/Translation Workbook

| Worksheet | Function |
|---|---|
| Screening Hit | Accepts user input for crystal density and reagent |
| | condition |
| Dilution Series | Generates a number of dilutions of a reagent variable. |
| Grid 1 | Generates a precipitant versus salt grid, while the |
| | original reagent pH remains fixed. |
| Grid 2 | Generates a grid of varying concentrations of |
| | precipitant and salt, at pH values around the original |
| | reagent pH. |
| Grid 3 | Generates a grid that varies pH against precipitant |
| | concentration, while salt concentration remains fixed. |
| Reagent Request | Provides part numbers and ordering information for |
| | stock reagents. |

Information about the hit reagent condition and an estimate of the crystal density is entered first in the Screening Hit worksheet. The conditions may be entered for example via a pull-down menu or via a variable code or number. As discussed above, crystal density (high, medium, low) is a measure of the number of crystals in the hit conditions from the small-volume experiment, see e.g. FIG. 1. The crystal density value alters the range and sampling interval used for each of the experiments in the worksheet. Based on the information entered in the Screening Hit worksheet, the database points to one of various worksheets that describe different experiments that can be run in a larger-volume experiment.

Nonlimiting examples of such worksheets labeled Dilution Series, Grid1, Grid 2, and Grid 3 are shown and described in more detail below.

The test values for variables in the worksheets are calculated based on adjustment factors that have been empirically formulated from previous experiments correlating crystal properties produced in small-volumes with crystal properties produced in larger-volumes. For high crystal density conditions, the nucleation rate in small volumes is high, and the reagent concentrations in each of the translation experiments are reduced by one adjustment factor to compensate for this. For medium crystal density, the nucleation rate is lower than that of a high density hit, and the reagent concentrations are adjusted with a smaller adjustment factor which provides a smaller sampling interval than for high density crystals. When crystal density is low, the nucleation rate is low in the small-volume experiment, and the database uses a smaller concentration adjust factor for the variables to form finer grids around the test levels of the variables of the small-volume experiment. Volumes on the worksheet can be adjusted based on the volumes of the devices used.

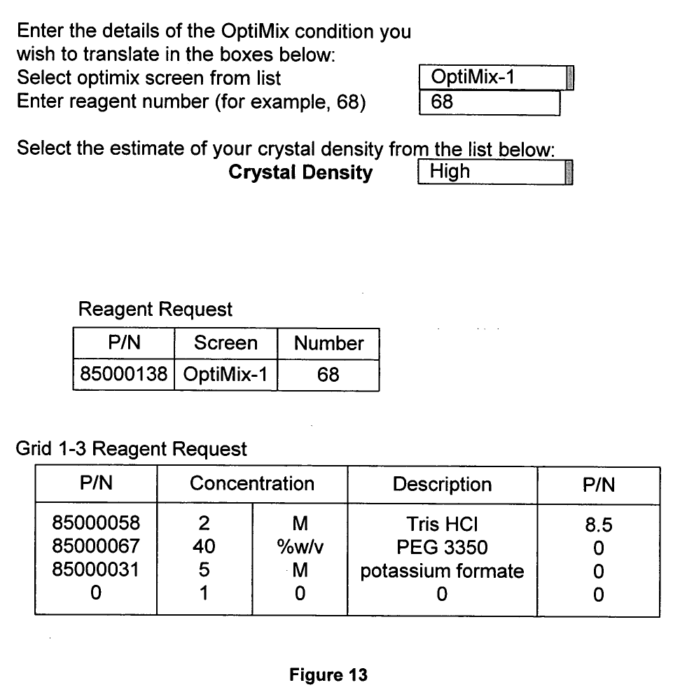

As shown in FIG. 13 when printed out, the Dilution Series worksheet and each of the three Grid worksheets provides two pages of information. Page 1 on each worksheet describes the starting reagent, its components, and details of the reagents in the suggested grid or dilution series. Page 2 on each worksheet, to the right of the Page 1 on each worksheet, provides a plating map for each of the variables of the condition. When each of the variables is loaded as described on Page 2 on each worksheet, the reagents mapped out by the worksheet can be prepared and positioned a larger-volume device such as a 96-well plate X-ray Chip. Each sheet generates a number of variables (e.g. twelve reagents) for running experiments against a target.

a. Dilution Series Worksheet

The Dilution Series worksheet provides a pipetting map for generating a simple series of dilutions of the starting reagent. At high crystal densities, the reagent is diluted in steps of 7.5% from the starting concentration. At medium crystal densities, the condition is diluted in steps of 5% from the starting concentration. At low crystal densities, the condition is diluted in steps of 2.5% from the starting reagent, as shown in Table 2.

TABLE 2
Percent Condition Dilution Series at 2.5% Intervals

| | 1 | 2 | 3 | 4 | 5 | 6 | 7 | 8 | 9 | 10 | 11 | 12 |
|---|---|---|---|---|---|---|---|---|---|---|---|---|
| A | 100 | 97.5 | 95 | | | | | | | | | |
| B | 92.5 | 90 | 87.5 | | | | | | | | | |
| C | 85 | 82.5 | 80 | | | | | | | | | |
| D | 77.5 | 75 | 72.5 | | | | | | | | | |

* Condition layout based on numbering system used on standard 96-well plate, with 12 columns, labeled 1-12, and 8 rows, labeled A-H. Any three columns on a 96-well plate may be used.

b. Grid 1 Worksheet

The Grid 1 worksheet calculates a simple grid of precipitant concentrations mixed with different salt concentrations, while maintaining the pH fixed at the original value of the hit reagent. At high crystal density, the grid is prepared with intervals of 15% initial concentration for the precipitant, starting at 100%; and intervals of 20% initial concentration of salt, starting at 100%. At medium crystal density, the interval for precipitant concentration is reduced to 10%, starting at 100%; the interval for salt changes to 15%, with a start concentration of 100%. At low crystal density, the precipitant concentration starts at 100%, and is reduced in steps of 5% initial concentration; the salt interval changes to 10%, with the maximum concentration at 100% initial concentration.

An example of high, medium, and low density grids calculated from a single reagent is shown in Table 3. The test levels of the variables in this example are 0.8M Ammonium acetate; 0.1M Morpholinoethanesulfonic acid (MES), pH 6.0; 20% polyethylene glycol (PEG). As the Low Density column in Table 3 illustrates, the initial precipitant concentration is 100% of the original precipitant, which in this example is 20% PEG. The initial salt concentration, also 100% of the original salt, is 0.8M. In the first row of three reagent wells in a 96-well reagent plate-A1, A2, A3—the precipitant concentration remains constant as the salt concentration is reduced in intervals of 10% of the original salt concentration. In the second row of three reagents-B1, B2, B3—the precipitant concentration is reduced by 5% of the initial concentration in all three wells while the changes in salt concentration used in the first row of reagents is repeated. Successive rows of reagents repeatedly reduce the precipitant concentration while limiting the changes in salt concentration.

TABLE 3
Examples of Grid 1 Designs for Translations Based on Crystal Densities
Observed on the X.96 Screening Chip [Starting condition is (0.8
M Ammonium acetate; 0.1 M MES, pH 6.0; 20% PEG 4600)]

| (96-well | | | | | | | | | |
|---|---|---|---|---|---|---|---|---|---|
| reagent | High Density | | | Medium Density | | | Low Density | | |
| plate) | pH | % Precip | Salt (M) | pH | % Precip | Salt (M) | pH | % Precip | Salt (M) |
| A1 | 6 | 20 | 0.8 | 6 | 20 | 0.8 | 6 | 20 | 0.8 |
| A2 | 6 | 20 | 0.64 | 6 | 20 | 0.68 | 6 | 20 | 0.72 |
| A3 | 6 | 20 | 0.48 | 6 | 20 | 0.56 | 6 | 20 | 0.64 |
| B1 | 6 | 17 | 0.8 | 6 | 18 | 0.8 | 6 | 19 | 0.8 |
| B2 | 6 | 17 | 0.64 | 6 | 18 | 0.68 | 6 | 19 | 0.72 |
| B3 | 6 | 17 | 0.48 | 6 | 18 | 0.56 | 6 | 19 | 0.64 |
| C1 | 6 | 14 | 0.8 | 6 | 16 | 0.8 | 6 | 18 | 0.8 |
| C2 | 6 | 14 | 0.64 | 6 | 16 | 0.68 | 6 | 18 | 0.72 |
| C3 | 6 | 14 | 0.48 | 6 | 16 | 0.56 | 6 | 18 | 0.64 |
| D1 | 6 | 11 | 0.8 | 6 | 14 | 0.8 | 6 | 17 | 0.8 |
| D2 | 6 | 11 | 0.64 | 6 | 14 | 0.68 | 6 | 17 | 0.72 |
| D3 | 6 | 11 | 0.48 | 6 | 14 | 0.56 | 6 | 17 | 0.64 |

c. Grid 2 Worksheet

The Grid 2 worksheet calculates a grid of precipitant concentrations mixed with different salt concentrations, and also varies the pH from the original value in the X.96 hit reagent. At high crystal density, the grid is prepared with intervals of 15% initial concentration for the precipitant, starting at 85%; and intervals of 25% initial concentration of salt, starting at 75%. At medium crystal density, the interval for precipitant concentration is reduced to 10%, starting at 90%; the interval for salt remains at 25%; but the start concentration changes to 100%. At low crystal density, the precipitant concentration starts at 100%, and is reduced in steps of 7.5% initial concentration; the salt interval stays at 25% with the maximum concentration at 100% initial concentration; the pH is varied in steps of 0.5 pH units. An example of the high, medium, and low density grids calculated from a single reagent from Grid 2 is shown in Table 4. The starting condition in this example is 0.8M Ammonium acetate; 0.1M MES, pH 6.0; 20% PEG.

TABLE 4
Examples of Grid 2 Designs for Translation Based on Crystal Densities
Observed on the X.96 Screening Chip [Starting condition is 0.8
M Ammonium acetate; 0.1 M MES, pH 6.0; 20% PEG 4600)]

| (96-well | | | | | | | | | |
|---|---|---|---|---|---|---|---|---|---|
| reagent | High Density | | | Medium Density | | | Low Density | | |
| plate) | pH | % Precip | Salt (M) | pH | % Precip | Salt (M) | pH | % Precip | Salt (M) |
| A1 | 5.5 | 17 | 0.4 | 5.5 | 18 | 0.6 | 5.5 | 20 | 0.6 |
| A2 | 6 | 17 | 0.6 | 6 | 18 | 0.8 | 6 | 20 | 0.8 |
| A3 | 6.5 | 17 | 0.2 | 6.5 | 18 | 0.4 | 6.5 | 20 | 0.4 |
| B1 | 5.5 | 14 | 0.4 | 5.5 | 16 | 0.6 | 5.5 | 18.5 | 0.6 |
| B2 | 6 | 14 | 0.6 | 6 | 16 | 0.8 | 6 | 18.5 | 0.8 |
| B3 | 6.5 | 14 | 0.2 | 6.5 | 16 | 0.4 | 6.5 | 18.5 | 0.4 |
| C1 | 5.5 | 11 | 0.4 | 5.5 | 14 | 0.6 | 5.5 | 17 | 0.6 |
| C2 | 6 | 11 | 0.6 | 6 | 14 | 0.8 | 6 | 17 | 0.8 |
| C3 | 6.5 | 11 | 0.2 | 6.5 | 14 | 0.4 | 6.5 | 17 | 0.4 |
| D1 | 5.5 | 8 | 0.4 | 5.5 | 12 | 0.6 | 5.5 | 15.5 | 0.6 |
| D2 | 6 | 8 | 0.6 | 6 | 12 | 0.8 | 6 | 15.5 | 0.8 |
| D3 | 6.5 | 8 | 0.2 | 6.5 | 12 | 0.4 | 6.5 | 15.5 | 0.4 |

As the Low Density column in Table 4 illustrates, the initial precipitant concentration is 100% of the original precipitant, which in this example is 20% PEG. The 10 salt concentration ranges from 100-60% of the original salt concentration, 0.8M. The original pH range varies ±0.5 pH units.

d. Grid 3 Worksheet

Grid 3 worksheet produces a grid that samples a broad range of pH (4.0-9.0) while varying the precipitant in a manner similar to that in Grid 2; and keeping the salt at a constant concentration, which is dependent upon the crystal density.

Reagents that contain calcium and magnesium are known to form salt crystals at higher pH values; for variables that contain these components the pH range is restricted to pH 4- 8. Zinc salts are also known to cause false positive crystallization at higher pH values; for variables containing zinc, the pH range is restricted to between pH 4-7.

An example of the high, medium, and low density grids calculated from a single reagent by Grid 3 is shown in Table 5. The starting condition in this example is 0.8M Ammonium acetate; 0.1M MES, pH 6.0; 20% PEG as found in reagent 82 of an Optimix-1 kit (Fluidigm Corp.). As the Low Density column in Table 5 illustrates, the initial precipitant concentration is 100% of the original precipitant, which in this example is 20% PEG, and is reduced in steps of 7.5% initial concentration. The salt concentration remains at 100% of the original salt concentration, 0.8M, while the pH range varies from 4.0-9.0.

TABLE 5
Examples of Grid 3 Designs for Translation Based on Crystal Densities
Observed on the X.96 Screening Chip [Starting condition is (0.8
M Ammonium acetate; 0.1 M MES, pH 6.0; 20% PEG 4600)]

| (96-well | | | | | | | | | |
|---|---|---|---|---|---|---|---|---|---|
| reagent | High Density | | | Medium Density | | | Low Density | | |
| plate) | pH | % Precip | Salt (M) | pH | % Precip | Salt (M) | pH | % Precip | Salt (M) |
| A1 | 4 | 17 | 0.6 | 4 | 18 | 0.8 | 4 | 20 | 0.8 |
| A2 | 4.5 | 11 | 0.6 | 4.5 | 14 | 0.8 | 4.5 | 17 | 0.8 |
| A3 | 5 | 14 | 0.6 | 5 | 16 | 0.8 | 5 | 18.5 | 0.8 |
| B1 | 5.5 | 17 | 0.6 | 5.5 | 18 | 0.8 | 5.5 | 20 | 0.8 |
| B2 | 6 | 11 | 0.6 | 6 | 14 | 0.8 | 6 | 17 | 0.8 |
| B3 | 6.5 | 14 | 0.6 | 6.5 | 16 | 0.8 | 6.5 | 18.5 | 0.8 |
| C1 | 7 | 17 | 0.6 | 7 | 18 | 0.8 | 7 | 20 | 0.8 |
| C2 | 7.5 | 11 | 0.6 | 7.5 | 14 | 0.8 | 7.5 | 17 | 0.8 |
| C3 | 8 | 14 | 0.6 | 8 | 16 | 0.8 | 8 | 18.5 | 0.8 |
| D1 | 8.5 | 17 | 0.6 | 8.5 | 18 | 0.8 | 8.5 | 20 | 0.8 |
| D2 | 9 | 11 | 0.6 | 9 | 14 | 0.8 | 9 | 17 | 0.8 |
| D3 | 9.5 | 14 | 0.6 | 9.5 | 16 | 0.8 | 9.5 | 18.5 | 0.8 |

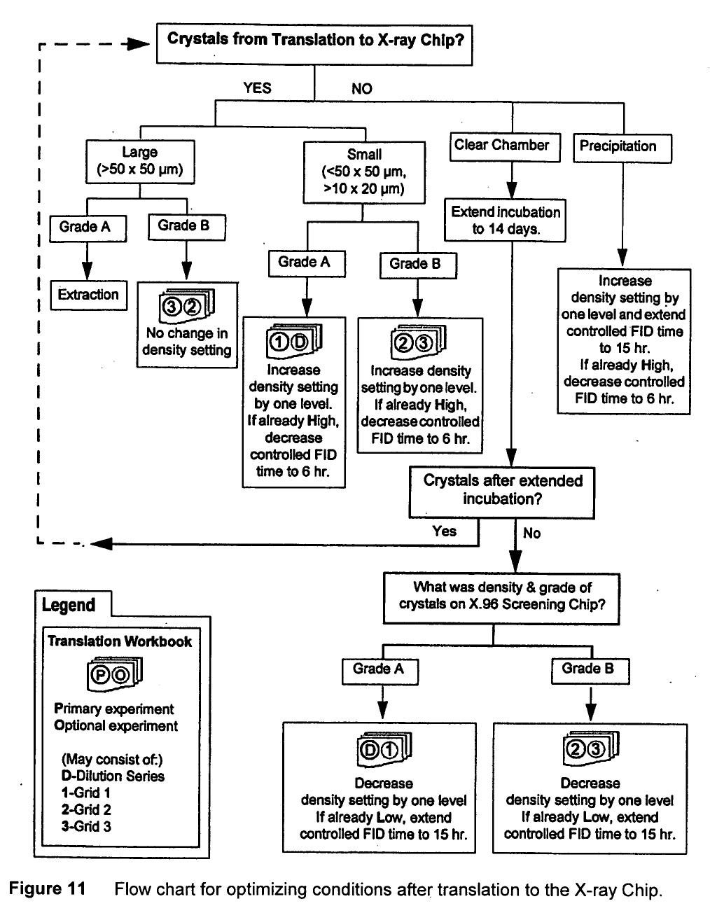

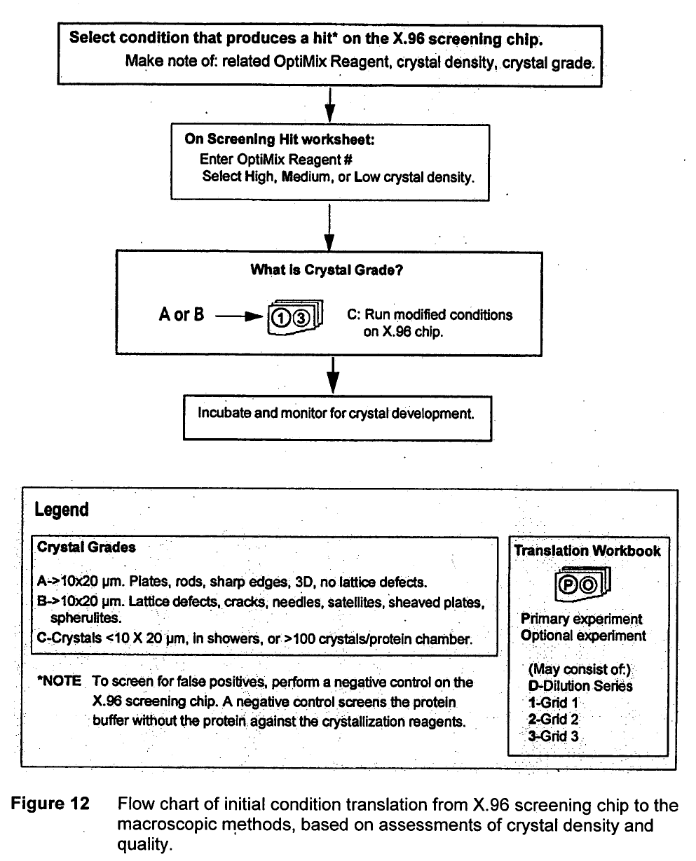

The grids described above can also be used for translation to macroscopic methods. As shown in the flow chart in FIG. 12, crystal found in the small-volume experiment are assessed for crystal density and crystal quality, as described above. Then the Grids corresponding to the appropriate crystal property are run.

If heavy precipitation is observed in all drops within 24 hours of setting up the experiment, it is recommended to prepare 3 dilutions of the initial reagents (80%, 60%, 40%), as shown in Table 6, and set up experiments against these diluted reagents.

Table 6 Preparing Dilutions of Initial Reagents

TABLE 6
Preparing Dilutions of Initial Reagents

| Final Dilution | Starting Reagent | |
|---|---|---|
| (%) | (μL) | Water (μL) |
| 80 | 80 | 20 |
| 60 | 60 | 40 |
| 40 | 40 | 60 |

Thus in one embodiment, the present invention provides a method of translating crystallization conditions from a small-volume experiment to a larger-volume experiment wherein when crystals of low density and medium quality are produced in the small-volume said adjusting is selected from the group consisting of:
a) adjusting the salt concentration in the higher volume experiment to a concentration in the range of about 50% to about 100% of the concentration from the low-volume experiment;
b) adjusting the precipitation reagent concentration in the higher volume experiment to a concentration in the range of about 75% to 100% of the concentration from the low-volume experiment; and
c) adjusting the pH in the high volume experiment in increments of 0.5 pH units of the pH of the low-volume experiment.

In another embodiment, the present invention provides a method of translating crystallization conditions from a small-volume experiment to a larger-volume experiment wherein when crystals of low density and medium quality are produced in the small-volume said adjusting is selected from the group consisting of;
a) adjusting the precipitation reagent concentration in the higher volume experiment to a concentration in the range of about 90% to 100% of the concentration from the low-volume experiment; and
b) adjusting the pH in the high volume experiment in increments of 0.5 pH units from the pH of the low-volume experiment.

Thus in another embodiment, the present invention provides a method of translating crystallization conditions from a small-volume experiment to a larger-volume experiment wherein when crystals of medium density and medium quality are produced in the small-volume said adjusting is selected from the group consisting of;
a) adjusting the salt concentration in the higher volume experiment to a concentration in the range of about 50% to about 100% of the concentration from the low-volume experiment;
b) adjusting the precipitation reagent concentration in the higher volume experiment to a concentration in the range of about 60% to 90% of the concentration from the low-volume experiment; and
c) adjusting the pH in the high volume experiment in increments of 0.5 pH units of the pH of the low-volume experiment.

Thus in another embodiment, the present invention provides a method of translating crystallization conditions from a small-volume experiment to a larger-volume experiment wherein when crystals of medium density and medium quality are produced in the small-volume said adjusting is selected from the group consisting of;
a) adjusting the precipitation reagent concentration in the higher volume experiment to a concentration in the range of about 70% to 90% of the concentration from the low-volume experiment; and
b) adjusting the pH in the high volume experiment in increments of 0.5 pH units of the pH of the low-volume experiment.

Thus in another embodiment, the present invention provides a method of translating crystallization conditions from a small-volume experiment to a larger-volume experiment wherein, wherein when crystals of high density and medium quality are produced in the small-volume said adjusting is selected from the group consisting of;
a) adjusting the salt concentration in the higher volume experiment to a concentration in the range of about 25% to about 75% of the concentration from the low-volume experiment;
b) adjusting the precipitation reagent concentration in the higher volume experiment to a concentration in the range of about 40% to 85% of the concentration from the low-volume experiment; and
c) adjusting the pH in the high volume experiment in increments of 0.5 pH units of the pH of the low-volume experiment.

Thus in another embodiment, the present invention provides a method of translating crystallization conditions from a small-volume experiment to a larger-volume experiment wherein when crystals of high density and medium quality are produced in the small-volume said adjusting is selected from the group consisting of;
a) adjusting the salt concentration in the higher volume experiment to a concentration in the range of about 75% of the concentration from the low-volume experiment;
b) adjusting the precipitation reagent concentration in the higher volume experiment to a concentration in the range of about 55% to 85% of the concentration from the low-volume experiment; and
c) adjusting the pH in the high volume experiment in increments of 0.5 pH units of the pH of the low-volume experiment.

Thus in another embodiment, the present invention provides a method of translating crystallization conditions from a small-volume experiment to a larger-volume experiment wherein, wherein when crystals of low density and high quality are produced in the small-volume said adjusting is selected from the group consisting of;
a) adjusting the salt concentration in the higher volume experiment to a concentration in the range of about 80% to about 100% of the concentration from the low-volume experiment; and
b) adjusting the precipitation reagent concentration in the higher volume experiment to a concentration in the range of about 70% to 100% of the concentration from the low-volume experiment.

Thus in another embodiment, the present invention provides a method of translating crystallization conditions from a small-volume experiment to a larger-volume experiment wherein, wherein when crystals of low density and high quality are produced in the small-volume the precipitation reagent concentration in the higher volume experiment is adjusted to a concentration in the range of about 85% to 100% of the concentration from the low-volume experiment.

Thus in another embodiment, the present invention provides a method of translating crystallization conditions from a small-volume experiment to a larger-volume experiment wherein, wherein when crystals of medium density and high quality are produced in the small-volume said adjusting is selected from the group consisting of;
a) adjusting the salt concentration in the higher volume experiment is adjusted to a concentration in the range of about 70% to about 100% of the concentration from the low-volume experiment; and
b) adjusting the precipitation reagent concentration in the higher volume experiment is adjusted to a concentration in the range of about 45% to 100% of the concentration from the low-volume experiment.

Thus in another embodiment, the present invention provides a method of translating crystallization conditions from a small-volume experiment to a larger-volume experiment wherein, wherein when crystals of medium density and high quality are produced in the small-volume the precipitation reagent concentration in the higher volume experiment is adjusted to a concentration in the range of about 70% to 100% of the concentration from the low-volume experiment.

Thus in another embodiment, the present invention provides a method of translating crystallization conditions from a small-volume experiment to a larger-volume experiment wherein, wherein when crystals of high density and high quality are produced in the small-volume said adjusting is selected from the group consisting of;
a) adjusting the salt concentration in the higher volume experiment to a concentration in the range of about 60% to about 100% of the concentration from the low-volume experiment; and
b) adjusting the precipitation reagent concentration in the higher volume experiment to a concentration in the range of about 30% to 100% of the concentration from the low-volume experiment.

Thus in another embodiment, the present invention provides a method of translating crystallization conditions from a small-volume experiment to a larger-volume experiment wherein, wherein when crystals of high density and high quality are produced in the small-volume the precipitation reagent concentration in the higher volume experiment is adjusted to a concentration in the range of about 50% to 100% of the concentration from the low-volume experiment.

The resolution of a grid may be selected based upon prior experience or by an embedded algorithm within the system. As the experimental data indicates that finer and finer resolution of grids may be in order, the system may then makes adjustments to the scope or resolution it will suggest in the subsequent grid so as to best optimize conditions for scale up. For example, if in the initial screen the nucleation rate is high, the system may suggest wider intervals between grid points so as to reduce the number of nucleation events in the scaled-up reaction. If the initial screen yielded a medium crystal density, then the sampling interval within the grid may be reduced in comparison to the high nucleation event screen. For low crystal densities where the nucleation rate is low, the grid may have a fine resolution to yield the best results.

In use, the system would contain information about the reagents used in the primary screening for crystallization conditions. After the primary screen, the reactions would be analyzed to identify which of the reagents used yielded crystals. For example, if a particular condition yielded the best crystals at a medium nucleation rate, that information would be entered into the spreadsheet to create the scale-up screen.

In another embodiment, the database comprises a reagent request worksheet which provides: a description of the stock solutions required for preparing the worksheet grids, part numbers, and information required to order the appropriate reagent stocks.

In another embodiment, the formulation information may be exported into an automated dispensing robot system to automatically prepare the reagent grid. Preferably, stock reagents would be supplied by a vendor that has already performed quality control experiments using the reagents in the microfluidic systems that are to be employed in with the reagent grids. The spreadsheet or computer/database may also make recommendations based on prior experimental data as to alternative stock reagent kits to use. If the computer system is connected to a network, such as the internet, the reagents may be automatically ordered so that supplies are maintained on-site to minimize delays in the advancement of the crystallization program.

FIG. 13 is a printout of a spreadsheet that represents how the foregoing spreadsheet may also appear and be used.

3. Further Optimizing Translation Experiments

Even when crystals are generated that allow the collection of diffraction data sets, they must also be of sufficiently high quality to calculate electron density maps to solve the structural problem under study. As with any experiment, initial results from a translation experiment may require refinement before the best results are obtained e.g. obtaining the best crystals for structure determination. This refinement may include: further screening of crystallization conditions; the inclusion of additives into the crystallization process; the use of alternative purification strategies; or manipulation of the macromolecule sample under investigation. The flow chart in FIG. 11 provides guidance for dealing with different outcomes of translation experiments that are run on a larger-volume device. In FIG. 11, the outcome from a translation experiment can be broadly divided into four major categories. 1) Large, single crystals are produced that can be extracted directly from the larger-scale device and used for diffraction data collection. 2) The crystals produced are difficult to extract from the larger-scale device due to their small size or fragile morphology. In this context, a small crystal is a crystal that is less than 50 μm on two sides. 3) There is precipitation, but no large crystal formation. 4) There is no crystal formation. If large, single crystals are produced, no further optimization is necessary.

If fragile or small crystals are produced, the crystallization conditions may be further refined in a larger-volume experiment to improve the crystals. In the case of small crystals, the concentrations of the variables are preferably further reduced. In the case of poor quality crystals (for example, sheaved or cracked crystals), the pH and the concentrations of the variables in the crystallization condition are further varied.

If precipitation is observed in many of the chambers, the same grid is rerun, with the density setting in the Translation Workbook increased by one value, as shown in FIG. 11. For example, if high density was selected for the initial grid, choose medium density for the subsequent grid. Precipitation in every chamber within the chip may indicate an enhanced sensitivity to a rapidly diffusing component-typically a component with low molecular weight-within the reagent. In this situation, its is recommended to run a new chip with the concentration of the stock salt solution reduced to 25% of the starting stock solution. In all other respects, the grid prepared should remain the same.

If no crystals appear in any chambers in the larger-volume device, the variable concentrations are too low. In this situation, the same grid is rerun with the density setting in the Translation Workbook reduced by one setting.

For example, if reagents for Grid 2 and Grid 3 were calculated at medium density and all chambers remained clear, the subsequent translation experiments would use Grid 2 and Grid 3 calculated at low density. If clear chambers were based on grids that were already calculated at low density, then the same reagents with a longer FID time would be run. For a chip run at room temperature, the FID time could be extended to 15 hours (e.g. using the X4 FID 4C script). For a chip run at 4° C., the FID time could be extended to about 25 hours.

In another aspect of the present invention, the database may be refined by an iterative process to improve the predictive nature of the database. Promising screening results can also be utilized as a basis for further screening focusing on a narrower spectrum of crystallization conditions, in a manner analogous to the use of standardized sparse matrix techniques. Moreover, the use of the database enables combinatorial screening of an array of different chemicals against a particular target or set of targets, a process that is difficult with either robotic or hand screening. This latter aspect is particularly important for optimizing initial successes generated by first-pass screens.

In another aspect of the present invention, the database would be in communication with an automated crystal detection imaging system that analyzes the outcome of screening and refinement experiments. By using the automated image analysis system to identify conditions that yielded crystal formation, condition space about those identified conditions could be further explored and the outcome comparatively analyzed.

The more factors considered when planning a scale up reaction, the higher the success rate that a crystal of a desired size will result from that condition. Although many factors may be understood, it is still possible that other unknown factors may come into play during scale up.

One way to address such unknown factors is to develop model systems for planning scale up reactions, the model systems being based wholly or in part by empirical observation. Using conventional protein crystallization assay systems, developing such empirical data may be laborious. However, by using microfluidic devices, such as those described by Hansen et al in U.S. patent application Ser. No. 11/006,522, filed Dec. 6, 2004; Ser. No. 09/724,784 filed Nov. 28, 2000; Ser. No. 10/997,714 filed Nov. 24, 2004; Ser. No. 60/525,245 filed Nov. 26, 2003 (including Appendix); Ser. No. 09/605,520, filed Jun. 27, 2000; U.S. patent Publication Nos. 20050019794, 20050062196, 20040115731, 20030061687, 20020145231, 20010020636 and International Patent Publication No. WO 01/32930, high-throughput analysis can be performed wherein large arrays of different conditions can be screened in parallel to characterize the differences between the calculated nucleation rate of crystallization from the actual rate, thus resulting in a constant that can be incorporated into the scale-up calculation to better achieve the desired rate of nucleation and thus the desired larger crystals suitable for use in x-ray diffraction experiments.

Other factors may likewise be altered to identify optimal scale-up conditions. For example, if the screening experiment yielded a condition having a first concentration of a crystallization reagent component, the scale-up screen may explore about that concentration to determine whether changes in the reaction volume are affected by changes in the concentration of that component.

In one embodiment, the concentration of a salt or other component present in the crystallization reagent solution may be varied about the concentration identified in the original screening reaction, however, the variant experiments would be conducted in the scale up reaction chamber rather than within an environment such as the original screening environment.

The following examples are intended to illustrate aspects of the invention. However, they are for illustration only and are not intended to limit the invention in any fashion.

### EXAMPLES

General Procedures

A set of test proteins were screened using Topaz 1.96 screening chips with OptiMix-1,-2, -3 and -4 (Fluidigm Corporation, South San Francisco) reagent sets to test for crystallization activity. The test proteins used to generate the scale factors were: alpha-lactalbumin, Glucose isomerase, Catalase, p97 AAA Atpase Domain1, Insulin. Reagents that resulted in crystal formation were identified and the crystallization results were classified on the basis of crystal quality and crystal density. Screening chip experiments were selected to cover a range of crystal densities and crystal qualities.

Major variables in the experiment were identified (precipitant concentration, salt concentration, temperature, pH, protein concentration). A full-factorial experiment was designed to test each of these variables to different concentrations, and experiments were carried out at larger-volumes. Experiments were assessed on the basis of crystal quality and crystal density, and major factors affecting the outcome of the experiment were identified (e.g. pH, precipitant, salt, etc.). On this basis, appropriate adjustment factors were identified using the results from the larger-volume crystallization experiments. The growth conditions that produced the best crystals were identified as those with the lowest crystal density and highest crystal grade. The values of the variables (e.g. pH, salt concentration, precipitant concentration) for these larger-volume crystal growth conditions were compared with the values of the same variables for the smaller-volume crystallization experiments for the translation of crystals from X.96 screening chips to X-ray chips, vapor diffusion and microbatch.

### Example 1

Crystallization of Myristoylated 22 kDa Guanylate Cyclase Activating Protein-] (cmGCAP-1)

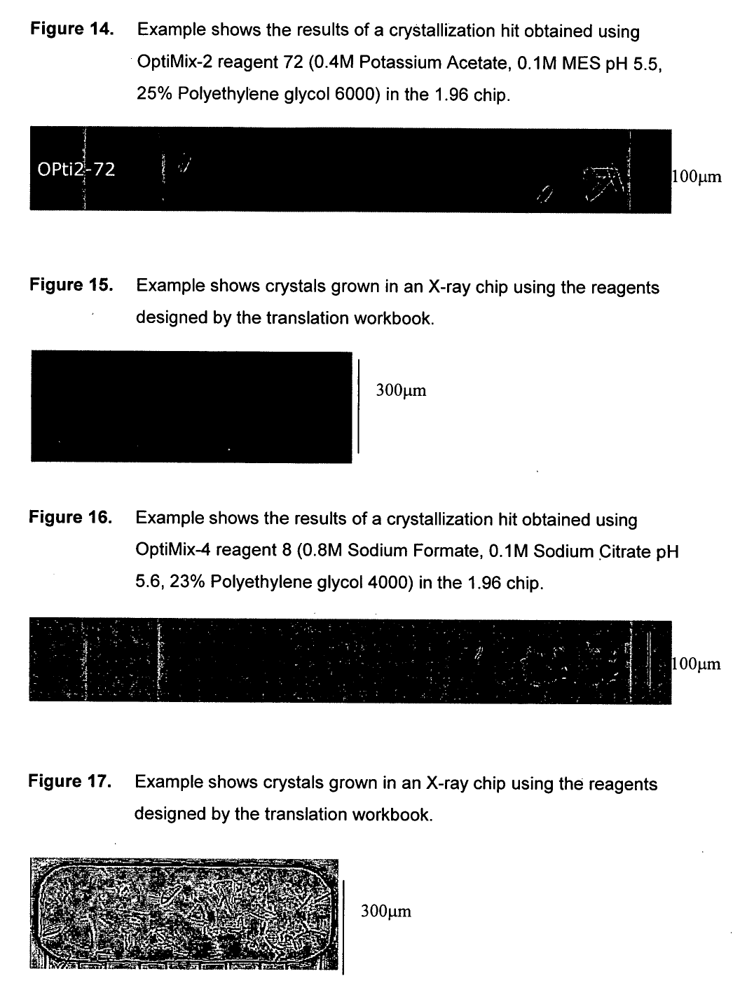

Crystallization hits were obtained in a number of reagents. Crystallization hits were selected to represent a variety of crystal density conditions. Reagents for translation were prepared using the formulae generated by the Translation Workbook, and a Topaz X-ray chip (Fluidigm Corporation) was set up according to the manufacturer's instructions. FIG. 14 shows the results of a crystallization hit obtained using OptiMix-2 reagent 72 (0.4M Potassium Acetate, 0.1M MES pH 5.5, 25% Polyethylene glycol 6000) in the 1.96 chip. This condition was classified as A-grade, low density, and Grid 1 was chosen as the appropriate grid to run for this experiment. FIG. 15 shows crystals grown in an X-ray chip using the reagents designed by the translation workbook. The crystals were extracted from the X-ray chip, and X-ray diffraction data were collected to 3.0 A at BL 12.3.1. at the Advanced Light Source at Lawrence Berkeley National Laboratory.

### Example 2

Crystallization of P97 AAA Atpase Domain 2.

Crystallization hits were obtained in a number of reagents. Crystallization hits were selected to represent a variety of crystal density conditions. Reagents for translation were prepared using the formulae generated by the Translation Workbook, and a Topaz X-ray chip (Fluidigm Corporation) was set up according to the manufacturer's instructions. FIG. 16 shows the results of a crystallization hit obtained using OptiMix-4 reagent 8 (0.8M Sodium Formate, 0.1M Sodium Citrate pH 5.6, 23% Polyethylene glycol 4000) in the 1.96 chip. This condition was classified as B-grade, high density, and Grid 2 was chosen as the appropriate grid to run for this experiment. FIG. 17 shows crystals grown in an X-ray chip using the reagents designed by the translation workbook.

### Example 3

Crystallization of P97 AAA Atpase Domain 1.

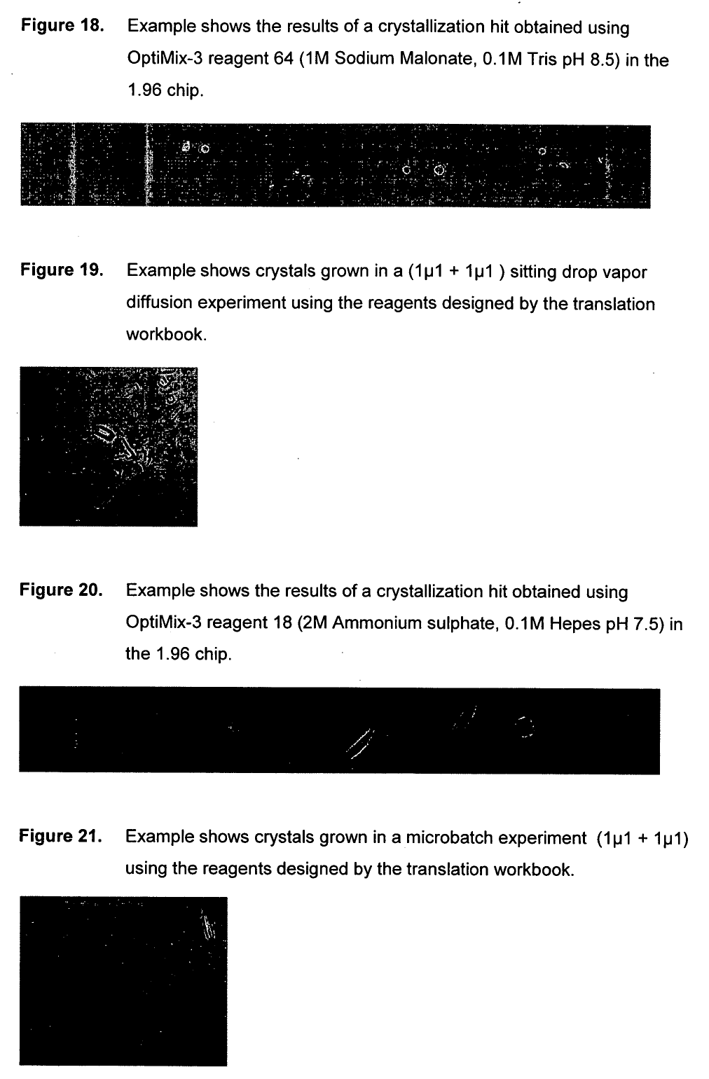

Crystallization hits were obtained in a number of reagents. Crystallization hits were selected to represent a variety of crystal density conditions. Reagents for translation were prepared using the formulae generated by the Translation Workbook, and sitting drop vapor diffusion experiments were set up in Greiner Crystal Quick sitting drop plates according to the “Guide to Translating Screening Conditions” (Fluidigm Corporation). FIG. 18 shows the results of a crystallization hit obtained using OptiMix-3 reagent 64 (1M Sodium Malonate, 0.1M Tris pH 8.5) in the 1.96 chip. Experimental formulations were calculated using the translation workbook. FIG. 19 shows crystals grown in a (1 μl+1 μl) sitting drop vapor diffusion experiment using the reagents designed by the translation workbook. Crystals from this drop were harvested and X-ray diffraction data were collected to 3.0 A at BL 12.3.1. at the Advanced Light Source at Lawrence Berkeley National Laboratory

### Example 4

Crystallization of Alpha-Lactalbumin.

Crystallization hits were obtained in a number of reagents. Crystallization hits were selected to represent a variety of crystal density conditions. Reagents for translation were prepared using the formulae generated by the Translation Workbook, and microbatch experiments were set up in Greiner microbatch plates according to the “Guide to Translating Screening Conditions” (Fluidigm Corporation). FIG. 20 shows the results of a crystallization hit obtained using OptiMix-3 reagent 18 (2M Ammonium sulphate, 0.1M Hepes pH 7.5) in the 1.96 chip. Experimental formulations were calculated using the translation workbook. FIG. 21 shows crystals grown in a microbatch experiment (1μl+1 μl) using the reagents designed by the translation workbook.

While the present invention has been described with reference to the specific embodiments thereof, it should be understood by those skilled in the art that various changes can be made and equivalents can be substituted without departing from the scope of the invention. In addition, many modifications can be made to adapt a particular situation, material, composition of matter, process, process step or steps, to achieve the benefits provided by the present invention without departing from the scope of the present invention. All such modifications are intended to be within the scope of the claims appended hereto.

All publications and patent documents cited herein are incorporated herein by reference as if each such publication or document was specifically and individually indicated to be incorporated herein by reference. Citation of publications and patent documents is not intended as an indication that any such document is pertinent prior art, nor does it constitute any admission as to the contents or date of the same.

What is claimed is:

1. A method of translating conditions from a small-volume to a larger-volume comprising:
(a) identifying one or more first test level(s) of one or more variable(s) that produce a first performance characteristic in a small volume;
(b) adjusting one or more level with an adjustment factor which factor which is based on the first performance characteristic produced in the small volume and the volume differences between the small volume and the larger volume; thereby producing one or more second test level(s) of one or more variable(s) for producing a second performance characteristic in a larger volume.

2. A method of producing a product in a large-volume comprising:
(a) identifying one or more test level(s) of one or more variable(s) in a small volume that produce a first performance characteristic;
(b) adjusting one or more test level(s) with an adjustment factor which is based on the first performance characteristic produced in the small volume and the volume difference between the small volume and a larger volume; thereby producing one or more second test level(s) of one or more variable(s);
(c) producing a product using said second test level(s) in a larger-volume.

3. The method of claim 1 further comprising:
performing a plurality of experiments that vary one or more test level(s) of one or more variable(s) to produce a performance characteristic.

4. A method of claim 1, wherein the ratio of the small-volume to large volume is from about 1:10 to about 1:1000.

5. A method of claim 1, wherein the small-volume is at most about 70 nanoliters and the larger-volume is at least about 75 nanoliters.

6. The method of claim 3, wherein said experiments are conducted in a device selected from the group consisting of a larger-volume device and a small-volume device.

7. The method of claim 6, wherein said device is a microfluidic device comprising a plurality of chambers.

8. The method of claim 6, wherein said large-volume device is selected from the group consisting of a microtiter plate, a vapor diffusion plate, a microbatch plate, a microdialysis chamber and a capillary tube.

9. The method of claim 1, wherein the test level is a concentration of a variable or a rate relating to a variable.

10. A method of formulating an adjustment factor for translating conditions from a small-volume to a larger-volume comprising:
(a) identifying one or more test level(s) of one or more variables that produce a first performance characteristic in a small volume for further analysis;
(b) identifying one or more test level(s) of one or more variables that produce a second performance characteristic in a large volume for further analysis;
(c) forming an analytical model for obtaining optimum values of the performance characteristic using the identified test levels of variables; and
(e) formulating an adjustment factor using the analytical model.

11. The method of claim 10, wherein the values are obtained from a plurality of experiments that vary one or more test level(s) of one or more variable(s) to produce a performance characteristic.

12. A method of translating crystallization conditions from a small-volume experiment to a larger-volume experiment comprising:
(a) identifying one or more first test level(s) of one or more variable(s) that produce a first crystal property in the small volume;
(b) adjusting one or more test level with an adjustment factor which factor which is based on the first performance characteristic produced in the small volume and the volume differences between the small volume and the larger volume; thereby producing one or more second test level(s) for producing a second crystal property in a larger volume.

13. A method of producing an x-ray quality crystal comprising:
(a) identifying one or more first test level(s) of one or more variable(s) that produce a first crystal property in a small-volume;
(b) adjusting one or more test level(s) with an adjustment factor which is based on the first crystal property produced in the small volume experiment and the volume difference between the small volume experiment and the larger volume experiment;
(c) producing a x-ray quality crystal using said second test level(s) in a larger-volume.

14. A method of claim 12, wherein the test level of a variable is selected from the group consisting of crystallization compound concentration, a precipitation reagent concentration, a pH modifying agent concentration, a salt concentration, a crystallization compound diffusion rate, a crystallization compound nucleation rate, a precipitation reagent diffusion rate, a pH modifying agent diffusion rate, a salt diffusion rate, and combinations thereof.

15. A method of claim 12, wherein the crystal property is selected from the group consisting of crystal density, crystal quality, crystal morphology, crystal size, and combinations thereof.

16. A method of claim 15, wherein the crystal density is selected from the group consisting of low density, medium density and high density; the crystal quality is selected from the group consisting of low quality, medium quality and high quality.

17. A method of claim 16, wherein when crystals of low density and medium quality are produced in the small-volume said adjusting is selected from the group consisting of:
a) adjusting the salt concentration in the higher volume experiment to a concentration in the range of about 50% to about 100% of the concentration from the low-volume experiment;
b) adjusting the precipitation reagent concentration in the higher volume experiment to a concentration in the range of about 75% to 100% of the concentration from the low-volume experiment; and
c) adjusting the pH in the high volume experiment in increments of 0.5 pH units of the pH of the low-volume experiment.

18. A method of claim 17, wherein when crystals of low density and medium quality are produced in the small-volume said adjusting is selected from the group consisting of;
a) adjusting the precipitation reagent concentration in the higher volume experiment to a concentration in the range of about 90% to 100% of the concentration from the low-volume experiment; and
b) adjusting the pH in the high volume experiment in increments of 0.5 pH units from the pH of the low-volume experiment.

19. A method of claim 16, wherein when crystals of medium density and medium quality are produced in the small-volume said adjusting is selected from the group consisting of;
a) adjusting the salt concentration in the higher volume experiment to a concentration in the range of about 50% to about 100% of the concentration from the low-volume experiment;
b) adjusting the precipitation reagent concentration in the higher volume experiment to a concentration in the range of about 60% to 90% of the concentration from the low-volume experiment; and
c) adjusting the pH in the high volume experiment in increments of 0.5 pH units of the pH of the low-volume experiment.

20. A method of claim 19, wherein when crystals of medium density and medium quality are produced in the small-volume said adjusting is selected from the group consisting of;
a) adjusting the precipitation reagent concentration in the higher volume experiment to a concentration in the range of about 70% to 90% of the concentration from the low-volume experiment; and
b) adjusting the pH in the high volume experiment in increments of 0.5 pH units of the pH of the low-volume experiment.

21. A method of claim 16, wherein when crystals of high density and medium quality are produced in the small-volume said adjusting is selected from the group consisting of;
a) adjusting the salt concentration in the higher volume experiment to a concentration in the range of about 25% to about 75% of the concentration from the low-volume experiment;
b) adjusting the precipitation reagent concentration in the higher volume experiment to a concentration in the range of about 40% to 85% of the concentration from the low-volume experiment; and
c) adjusting the pH in the high volume experiment in increments of 0.5 pH units of the pH of the low-volume experiment.

22. A method of claim 21, wherein when crystals of high density and medium quality are produced in the small-volume said adjusting is selected from the group consisting of;
a) adjusting the salt concentration in the higher volume experiment to a concentration in the range of about 75% of the concentration from the low-volume experiment;
b) adjusting the precipitation reagent concentration in the higher volume experiment to a concentration in the range of about 55% to 85% of the concentration from the low-volume experiment; and
c) adjusting the pH in the high volume experiment in increments of 0.5 pH units of the pH of the low-volume experiment.

23. A method of claim 16, wherein when crystals of low density and high quality are produced in the small-volume said adjusting is selected from the group consisting of;
a) adjusting the salt concentration in the higher volume experiment to a concentration in the range of about 80% to about 100% of the concentration from the low-volume experiment; and
b) adjusting the precipitation reagent concentration in the higher volume experiment to a concentration in the range of about 70% to 100% of the concentration from the low-volume experiment.

24. A method of claim 23, wherein when crystals of low density and high quality are produced in the small-volume the precipitation reagent concentration in the higher volume experiment is adjusted to a concentration in the range of about 85% to 100% of the concentration from the low-volume experiment.

25. A method of claim 16, wherein when crystals of medium density and high quality are produced in the small-volume said adjusting is selected from the group consisting of;
a) adjusting the salt concentration in the higher volume experiment is adjusted to a concentration in the range of about 70% to about 100% of the concentration from the low-volume experiment; and
b) adjusting the precipitation reagent concentration in the higher volume experiment is adjusted to a concentration in the range of about 45% to 100% of the concentration from the low-volume experiment.

26. A method of claim 25, wherein when crystals of medium density and high quality are produced in the small-volume the precipitation reagent concentration in the higher volume experiment is adjusted to a concentration in the range of about 70% to 100% of the concentration from the low-volume experiment.

27. A method of claim 16, wherein when crystals of high density and high quality are produced in the small-volume said adjusting is selected from the group consisting of;
a) adjusting the salt concentration in the higher volume experiment to a concentration in the range of about 60% to about 100% of the concentration from the low-volume experiment; and
b) adjusting the precipitation reagent concentration in the higher volume experiment to a concentration in the range of about 30% to 100% of the concentration from the low-volume experiment.

28. A method of claim 27, wherein when crystals of high density and high quality are produced in the small-volume the precipitation reagent concentration in the higher volume experiment is adjusted to a concentration in the range of about 50% to 100% of the concentration from the low-volume experiment.

29. A computer implemented method of translating conditions from a small-volume to a larger-volume comprising:
(a) identifying one or more test level(s) of one or more variables that produce a first performance characteristic in a small volume for further analysis;
(b) identifying one or more test level(s) of one or more variables that produce a second performance characteristic in a large volume for further analysis;
(c) recording the values of the test levels, variables and performance characteristics at a particular volume;
(d) forming an analytical model for obtaining optimum values using the recorded values; and
(e) formulating an adjustment factor using the analytical model thereby producing a second set of test levels for producing a second performance characteristic in a larger volume.

30. The method of claim 29, wherein the recording is done in a database.

31. The method of claim 29, wherein the values are obtained from a plurality of experiments that vary one or more test level(s) of one or more variable(s) to produce a performance characteristic.

32. The method of claim 31 wherein the experiments are protein crystallization screening assays.

33. A database formed by the method according to claim 29.

34. A system comprising:
a database of claim 33 and
a computer apparatus having access to said database capable of analyzing the outcome of small-volume and larger volume experiments.

35. A system of claim 34, wherein the system is in communication with an automated crystal detection imaging system.

36. A system of claim 34, wherein the database is in communication with a robotic liquid dispensing system capable of preparing or dispensing new test values of variables.

37. A method for using the system of claim 34 comprising:
(a) identifying in the database one or more test level(s) of one or more variable(s) in a small volume using the computer;
(b) obtaining one or more second test level(s) of one or more variable(s) for a larger-volume;
(c) producing a product using said second test level(s) in a larger-volume.
